(19) United States
(12) Patent Application Publication
(10) Pub. No.: US 2023/0407003 A1
(43) Pub. Date: Dec. 21, 2023

(54) FLUORINE-CONTAINING ETHER COMPOUND, SURFACE TREATMENT AGENT, FLUORINE-CONTAINING ETHER COMPOSITION, COATING LIQUID, ARTICLE, METHOD FOR PRODUCING ARTICLE, AND COMPOUND

(71) Applicant: AGC Inc., Tokyo (JP)

(72) Inventors: Motoshi AOYAMA, Chiyoda-ku (JP); Makoto UNO, Chiyoda-ku (JP)

(73) Assignee: AGC Inc., Tokyo (JP)

(21) Appl. No.: 18/456,804

(22) Filed: Aug. 28, 2023

Related U.S. Application Data
(63) Continuation of application No. PCT/JP2022/008843, filed on Mar. 2, 2022.

(30) Foreign Application Priority Data
Mar. 5, 2021 (JP) 2021-034907

(51) Int. Cl. C08G 65/00; C08G 65/336; C09D 171/00

(52) U.S. Cl. CPC C08G 65/007; C08G 65/336; C09D 171/00; C08G 2650/04

(57) ABSTRACT

There is provided a fluorine-containing ether compound excellent in light fastness, a surface treatment agent, a fluorine-containing ether composition, a coating liquid, an article having a surface layer excellent in light fastness, a method for producing the same, and a compound useful as a raw material of the fluorine-containing ether compound excellent in light fastness.

A fluorine-containing ether compound represented by the following general formula (A).

(R¹R²C═CR³-L¹-)ₙ₁Q¹-R^{f}-Q²(-T)ₙ₂ Formula (A)
where, each reference sign in the formula (A) is as described in the specification.

### INCORPORATION BY REFERENCE

This application is based upon and claims the benefit of priority from Japanese Patent Application 2021-034907 filed on Mar. 5, 2021, and PCT application No. PCT/JP2022/008843 filed on Mar. 2, 2022, the disclosure of which is incorporated herein in its entirety by reference.

### BACKGROUND

The present invention relates to a fluorine-containing ether compound, a surface treatment agent, a fluorine-containing ether composition, a coating liquid, an article, a method for producing an article, and a compound.

Fluorine-containing ether compounds having a fluorine atom are excellent in various properties such as low refractive index, low dielectric constant, water/oil repellency, heat resistance, chemical resistance, chemical stability, and transparency, and are used in various fields such as electric and electronic materials, semiconductor materials, optical materials, and surface treatment agents.

For example, a fluorine-containing ether compound having a perfluoropolyether chain and a hydrolyzable silyl group is suitably used as a surface treatment agent because a surface layer exhibiting high lubricity, water/oil repellency, and the like can be formed on the surface of a substrate. The surface treatment agents containing the fluorine-containing ether compound are used for applications in which it is required to maintain performance (friction resistance) in which water/oil repellency is less likely to decrease even when the surface layer is repeatedly rubbed with a finger and performance (fingerprint dirt removability) in which a fingerprint attached to the surface layer can be easily removed by wiping, for a long period of time, for example, as a surface treatment agent for a member constituting a surface touched by a finger of a touch panel, a spectacle lens, and a display of a wearable terminal.

As a fluorine-containing ether compound capable of forming a surface layer excellent in friction resistance and fingerprint dirt removability on a surface of a substrate, a fluorine-containing ether compound having a perfluoropolyether chain and a hydrolyzable silyl group has been proposed (Japanese Unexamined Patent Application Publication No. 2016-037541 and International Patent Publication No. WO 2017/038830).

### SUMMARY

As described above, the fluorine-containing ether compound is useful as a surface treatment agent for imparting the various physical properties, and there is an increasing demand for a fluorine-containing ether compound that can be used under various environments. The present inventors conducted studies for the purpose of further improving light fastness.

The present invention solves the above problems, and an object thereof is to provide a fluorine-containing ether compound excellent in light fastness, a surface treatment agent, a fluorine-containing ether composition, a coating liquid, an article having a surface layer excellent in light fastness, a method for producing the same, and a compound useful as a raw material of the fluorine-containing ether compound excellent in light fastness.

The present invention relates to a fluorine-containing ether compound having the following configurations [1] to [7], a surface treatment agent, a fluorine-containing ether composition, a coating liquid, an article, a method for producing an article, and a compound.

[1] A fluorine-containing ether compound represented by the following general formula (A).

(R¹R²C═CR³-L¹-)ₙ₁Q¹-R^{f}-Q²(-T)ₙ₂ Formula (A)
where
R¹ and R² are each independently a hydrogen atom or an alkyl group which may have a substituent, and when there are a plurality of R¹'s and R²'s, the plurality of R¹'s and R²'s may be the same as or different from each other,
R³ is a hydrogen atom, a halogen atom, or an alkyl group which may have a substituent, and when there are a plurality of R³'s, the plurality of R³'s may be the same as or different from each other,
L¹ is an oxygen atom or CR⁴R⁵, and when there are a plurality of L¹'s, the plurality of L¹'s may be the same as or different from each other,
R⁴ and R⁵ are each independently a hydrogen atom, a halogen atom, or an alkyl group which may have a substituent, and when there are a plurality of R⁴'s and R⁵'s, the plurality of R⁴'s and R⁵'s may be the same as or different from each other,
R^{f} is a polyfluoropolyether chain,
Q¹ is an (n1+1)-valent linking group,
Q² is an (n2+1)-valent linking group,
T is Si(—R⁶)₃₋ₐ(—R⁷)ₐ, and when there are a plurality of T's, the plurality of T's may be the same as or different from each other,
R⁶ is a hydrogen atom or a hydrocarbon group, and when there are a plurality of R⁶'s, the plurality of R⁶'s may be the same as or different from each other,
R⁷ is a hydrolyzable group or a hydroxyl group, and when there are a plurality of R⁷'s, the plurality of R⁷'s may be the same as or different from each other,
a is an integer of 1 to 3,
n1 is an integer of 1 to 20, and
n2 is an integer of 1 to 20.

[2] A surface treatment agent containing the fluorine-containing ether compound according to [1].

[3] A fluorine-containing ether composition containing the fluorine-containing ether compound according to [1] and another fluorine-containing ether compound.

[4] A coating liquid containing the fluorine-containing ether compound according to [1] or the fluorine-containing ether composition according to [3], and a liquid medium.

[5] An article having a surface layer formed from the fluorine-containing ether compound according to [1] or the fluorine-containing ether composition according to [3].

[6] A method for producing an article, including: forming a surface layer by a dry coating method or a wet coating method using the fluorine-containing ether compound according to [1], the surface treatment agent according to [2], the fluorine-containing ether composition according to [3], or the coating liquid according to [4].

[7] A compound represented by the following general formula (B).

(R¹R²C═CR³-L¹-)ₙ₁Q¹-R^{f}-Q¹²(-L¹¹-R¹³C═CR¹²R¹¹)ₙ₂ Formula (B)
where
R¹ and R² are each independently a hydrogen atom or an alkyl group which may have a substituent, and when there are a plurality of R¹'s and R²'s, the plurality of R¹'s and R²'s may be the same as or different from each other,
R³ is a hydrogen atom, a halogen atom, or an alkyl group which may have a substituent, and when there are a plurality of R³'s, the plurality of R³'s may be the same as or different from each other,
L¹ is an oxygen atom or CR⁴R⁵, and when there are a plurality of L¹'s, the plurality of L¹'s may be the same as or different from each other,
R⁴ and R⁵ are each independently a hydrogen atom, a halogen atom, or an alkyl group which may have a substituent, and when there are a plurality of R⁴'s and R⁵'s, the plurality of R⁴'s and R⁵'s may be the same as or different from each other,
R¹¹ and R¹² are each independently a hydrogen atom or an alkyl group which may have a substituent, and when there are a plurality of R¹¹'s and R¹²'s, the plurality of R¹¹'s and R¹²'s may be the same as or different from each other,
R¹³ is a hydrogen atom, a halogen atom, or an alkyl group which may have a substituent, and when there are a plurality of R¹³'s, the plurality of R¹³'s may be the same as or different from each other,
L¹¹ is an oxygen atom or CR¹⁴R¹⁵, and when there are a plurality of L¹¹'s, the plurality of L¹¹'s may be the same as or different from each other,
R¹⁴ and R¹⁵ are each independently a hydrogen atom, a halogen atom, or an alkyl group which may have a substituent, and when there are a plurality of R¹⁴'s and R¹⁵'s, the plurality of R¹⁴'s and R¹⁵'s may be the same as or different from each other,
R¹R²C═CR³-L¹- and R¹¹R¹²C═CR¹³-L¹¹- are different structures,
R^{f} is a polyfluoropolyether chain,
Q¹ is an (n1+1)-valent linking group,
Q¹² is an (n2+1)-valent linking group,
n1 is an integer of 1 to 20, and
n2 is an integer of 1 to 20.

The above and other objects, features and advantages of the present disclosure will become more fully understood from the detailed description given hereinbelow which are given by way of illustration only, and thus are not to be considered as limiting the present disclosure.

The present invention is to provide a fluorine-containing ether compound excellent in light fastness, a surface treatment agent, a fluorine-containing ether composition, a coating liquid, an article having a surface layer excellent in light fastness, a method for producing the same, and a compound useful as a raw material of the fluorine-containing ether compound excellent in light fastness.

### BRIEF DESCRIPTION OF THE DRAWINGS

FIG. 1 is a schematic cross-sectional view showing an example of an article of the present invention.

### DESCRIPTION OF EMBODIMENTS

The meanings of the following terms in the present specification are as follows.

In the present specification, the compound represented by the formula (A) is referred to as a compound (A). The same applies to compounds represented by other formulae and the like.

The perfluoroalkyl group means a group in which all hydrogen atoms of the alkyl group are substituted with fluorine atoms. In addition, the fluoroalkyl group is a generic term for a combination of a partial fluoroalkyl group and a perfluoroalkyl group. The partial fluoroalkyl group is an alkyl group in which one or more hydrogen atoms are substituted with a fluorine atom and which has one or more hydrogen atoms. That is, the fluoroalkyl group is an alkyl group having one or more fluorine atoms.

“to” indicating a numerical range means that the numerical values stated before and after “to” are included as a lower limit value and an upper limit value.

The “reactive silyl group” is a generic term for a hydrolyzable silyl group and a silanol group (Si—OH).

The “hydrolyzable silyl group” means a group capable of forming a silanol group by a hydrolysis reaction.

The “surface layer” means a layer formed on the surface of the substrate.

The “molecular weight” of the polyfluoropolyether chain is a number average molecular weight calculated by determining the number (average value) of oxyfluoroalkylene units based on the terminal group by ¹H-NMR and ¹⁹F-NMR.

### [Fluorine-Containing Ether Compound]

The fluorine-containing ether compound (hereinafter also referred to as “the present compound”) of the present invention is a compound represented by the following general formula (A).

(R¹R²C═CR³-L¹-)ₙ₁Q¹-R^{f}-Q²(-T)ₙ₂ Formula (A)
where
R¹ and R² are each independently a hydrogen atom or an alkyl group which may have a substituent, and when there are a plurality of R¹'s and R²'s, the plurality of R¹'s and R²'s may be the same as or different from each other,
R³ is a hydrogen atom, a halogen atom, or an alkyl group which may have a substituent, and when there are a plurality of R³'s, the plurality of R³'s may be the same as or different from each other,
L¹ is an oxygen atom or CR⁴R⁵, and when there are a plurality of L¹'s, the plurality of L¹'s may be the same as or different from each other,
R⁴ and R⁵ are each independently a hydrogen atom, a halogen atom, or an alkyl group which may have a substituent, and when there are a plurality of R⁴'s and R⁵'s, the plurality of R⁴'s and R⁵'s may be the same as or different from each other,
R^{f} is a polyfluoropolyether chain,
Q¹ is an (n1+1)-valent linking group,
Q² is an (n2+1)-valent linking group,
T is Si(—R⁶)₃₋ₐ(—R⁷)ₐ, and when there are a plurality of T's, the plurality of T's may be the same as or different from each other,
R⁶ is a hydrogen atom or a hydrocarbon group, and when there are a plurality of R⁶'s, the plurality of R⁶'s may be the same as or different from each other,
R⁷ is a hydrolyzable group or a hydroxyl group, and when there are a plurality of R⁷'s, the plurality of R⁷'s may be the same as or different from each other,
a is an integer of 1 to 3,
n1 is an integer of 1 to 20, and
n2 is an integer of 1 to 20.

The present compound roughly has a structure of “olefin-linking group-polyfluoropolyether chain-linking group-reactive silyl group”.

Since the reactive silyl group is strongly chemically bonded to the substrate, the surface layer formed using the present compound is excellent in friction resistance.

Since the present compound has a polyfluoropolyether chain “R_{f}”, the fingerprint dirt removability of the surface layer is excellent.

In addition, in the present compound constituting the surface layer, an olefin is disposed in the vicinity of the interface on the opposite side to the substrate-side interface. Since the olefin has a radical trapping ability, generation of radicals due to ultraviolet rays or the like is suppressed, and the olefin is excellent in stability to light.

As described above, the present compound is useful as a surface treatment agent capable of forming a surface layer having excellent light fastness and excellent fingerprint removability and water/oil repellency. In addition, the present compound can be used in combination with another fluorine-containing ether compound, and the light fastness of the surface layer is improved as compared with the case where the other fluorine-containing ether compound is used alone.

R¹ and R² are each independently a hydrogen atom or an alkyl group which may have a substituent. The alkyl group may be a linear alkyl group or a branched alkyl group. The number of carbon atoms in the alkyl group is preferably 1 to 6, and more preferably 1 to 3. Examples of the substituent that the alkyl group may have include a halogen atom, an alkoxy group, and a hydroxyl group. Examples of the halogen atom include a fluorine atom, a chlorine atom, a bromine atom, and an iodine atom, and a fluorine atom, a chlorine atom, and a bromine atom are preferable, and a fluorine atom is more preferable from the viewpoint of water/oil repellency and fingerprint removability when used as a surface layer. The alkoxy group is preferably an alkoxy group having 1 to 6 carbon atoms which may have a substituent. Specific examples of the alkoxy group include a methoxy group, an ethoxy group, and a butoxy group. Examples of the substituent that the alkoxy group may have include halogen atoms, and among them, a fluorine atom, a chlorine atom, and a bromine atom are preferable, and a fluorine atom is more preferable from the viewpoint of water/oil repellency and fingerprint removability as a surface layer.

Among R¹ and R², a hydrogen atom, an alkyl group having 1 to 6 carbon atoms, or a fluoroalkyl group having 1 to 6 carbon atoms is preferable from the viewpoint of ease of synthesis and the like.

R³ is a hydrogen atom, a halogen atom, or an alkyl group which may have a substituent. Examples of the halogen atom in R³ include a fluorine atom, a chlorine atom, a bromine atom, and an iodine atom, and a fluorine atom, a chlorine atom, and a bromine atom are preferable, and a fluorine atom is more preferable from the viewpoint of water/oil repellency and fingerprint removability when used as a surface layer. The alkyl group which may have a substituent in R³ is the same as those in R¹ and R², and preferred embodiments are also the same.

L¹ is an oxygen atom or CR⁴R⁵. R⁴ and R⁵ are each independently a hydrogen atom, a halogen atom, or an alkyl group which may have a substituent. Halogen atoms in R⁴ and R⁵ are the same as those in the R³, and preferred embodiments are also the same. In addition, the alkyl group which may have a substituent in R⁴ and R⁵ is the same as those in R¹ and R², and preferred embodiments are also the same.

Specific examples of R¹R²C═CR³-L¹- include the following structures.
CH₂═CH—CH₂—, CH₂═CH—CF₂—, CH₂═CF—CF₂—, CF₂═CF—CF₂—, CF₃CF═CF—CF₂—, (CF₃—)₂C═CF—CF₂—, CF₃CF₂CF═CF—CF₂—, CF₃CF₂CF₂CF═CF—CF₂—, CF₂═C(—CF₃)—CF₂—, CF₂═CF—C(—CF₃)F—, CH₂═CH—O—, CH₂═CF—O—, CF₂═CF—O—, CF₃CF═CF—O—, (CF₃—)₂C═CF—O—, CF₃CF₂CF═CF—O—, CF₃CF₂CF₂CF═CF—O—, CF₂═C(—CF₃)—O—.

The number n1 of R¹R²C═CR³-L¹- in one molecule of the compound (A) may be 1 to 20, and n1 is preferably 1 to 12 and more preferably 1 to 6 from the viewpoint of ease of synthesis, ease of handling of the compound (A), and the like.

When there are two or more R¹R²C═CR³-L¹- in one molecule of the compound (A), the R¹R²C═CR³-L¹- may have the same structure as or different structures from each other.

R^{f} represents a divalent polyfluoropolyether chain. The polyfluoropolyether chain in R^{f} preferably has a structure represented by the following formula (F1).

—(O)ₘ₀—[(R^{f1}O)ₘ₁(R^{f2}O)ₘ₂(R^{f3}O)ₘ₃(R^{f4}O)ₘ₄(R^{f3}O)ₘ₅(R^{f6}O)ₘ₆]—(R^{f7})ₘ₇— Formula (F1)
where
R^{f1} is a fluoroalkylene group having 1 carbon atom,
R^{f2} is a fluoroalkylene group having 2 carbon atoms,
R^{f3} is a fluoroalkylene group having 3 carbon atoms,
R^{f4} is a fluoroalkylene group having 4 carbon atoms,
R^{f5} is a fluoroalkylene group having 5 carbon atoms,
R^{f6} is a fluoroalkylene group having 6 carbon atoms, R^{f7} is a fluoroalkylene group having 1 to 6 carbon atoms,
m0 is 0 or 1,
m1, m2, m3, m4, m5, and m6 each independently represent an integer of 0 or 1 or more, and
m7 is an integer of 0 or 1, and m1+m2+m3+m4+m5+m6 is an integer of 1 to 200.

Note that the bonding order of (R^{f1}O) to (R^{f6}O) in the formula (F1) is random.

m1 to m6 in the formula (F1) represent the number of (R^{f1}O) to (R^{f6}O), respectively, and do not represent arrangement. For example, (R^{f5}O)ₘ₅ represents that the number of (R^{f5}O) is m5, and does not represent a block arrangement structure of (R^{f5}O)ₘ₅. Similarly, the order of description of (R^{f1}O) to (R^{f6}O) does not represent the bonding order of the respective units.

When m7 is 0, one terminal of R^{f} bonded to Q² is —O—. When m7 is 1, one terminal of R^{f} bonded to Q² is a carbon atom (carbon atom at the terminal of R^{f7}). In addition, when m0 is 1, one terminal of R^{f} bonded to Q¹ is —O—. When m0 is one terminal of R^{f} bonded to Q¹ is a carbon atom (carbon atom at any terminal of R^{f1} to R^{f7}). Note that m0 and m7 are each independently 0 or 1.

In addition, the fluoroalkylene group having 3 to 6 carbon atoms may be a linear fluoroalkylene group or a fluoroalkylene group having a branched or ring structure.

Specific examples of R^{f1} include —CF₂— and —CHF—.

Specific examples of R^{f2} include —CF₂CF₂—, —CHFCF₂—, —CHFCHF—, —CH₂CF₂—, and —CH₂CHF—.

Specific examples of R^{f3} include —CF₂CF₂CF₂—, —CF₂CHFCF₂—, —CF₂CH₂CF₂—, —CHFCF₂CF₂—, —CHFCHFCF₂—, —CHFCHFCHF—, —CHFCH₂CF₂—, —CH₂CF₂CF₂—, —CH₂CHFCF₂—, —CH₂CH₂CF₂—, —CH₂CF₂CHF—, —CH₂CHFCHF—, —CH₂CH₂CHF—, —CF(CF₃)—CF₂—, —CF(CHF₂)—CF₂—, —CF(CH₂F)—CF₂—, —CF(CH₃)—CF₂—, —CF(CF₃)—CHF—, —CF(CHF₂)—CHF—, —CF(CH₂F)—CHF—, —CF(CH₃)—CHF—, —CF(CF₃)—CH₂—, —CF(CHF₂)—CH₂—, —CF(CH₂F)—CH₂—, —CF(CH₃)—CH₂—, —CH(CF₃)—CF₂—, —CH(CHF₂)—CF₂—, —CH(CH₂F)—CF₂—, —CH(CH₃)—CF₂—, —CH(CF₃)—CHF—, —CH(CHF₂)—CHF—, —CH(CH₂F)—CHF—, —CH(CH₃)—CHF—, —CH(CF₃)—CH₂—, —CH(CHF₂)—CH₂—, and —CH(CH₂F)—CH₂—.

Specific examples of R^{f4} include —CF₂CF₂CF₂CF₂—, —CHFCF₂CF₂CF₂—, —CH₂CF₂CF₂CF₂—, —CF₂CHFCF₂CF₂—, —CHFCHFCF₂CF₂—, —CH₂CHFCF₂CF₂—, —CF₂CH₂CF₂CF₂—, —CHFCH₂CF₂CF₂—, —CH₂CH₂CF₂CF₂—, —CHFCF₂CHFCF₂—, —CH₂CF₂CHFCF₂—, —CF₂CHFCHFCF₂—, —CHFCHFCHFCF₂—, —CH₂CHFCHFCF₂—, —CF₂CH₂CHFCF₂—, —CHFCH₂CHFCF₂—, —CH₂CH₂CHFCF₂—, —CF₂CH₂CH₂CF₂—, —CHFCH₂CH₂CF₂—, —CH₂CH₂CH₂CF₂—, —CHFCH₂CH₂CHF—, —CH₂CH₂CH₂CHF—, and -cycloC₄F₆—.

Specific examples of R^{f5} include —CF₂CF₂CF₂CF₂CF₂—, —CHFCF₂CF₂CF₂CF₂—, —CH₂CHFCF₂CF₂CF₂—, —CF₂CHFCF₂CF₂CF₂—, —CHFCHFCF₂CF₂CF₂—, —CF₂CH₂CF₂CF₂CF₂—, —CHFCH₂CF₂CF₂CF₂—, —CH₂CH₂CF₂CF₂CF₂—, —CF₂CF₂CHFCF₂CF₂—, —CHFCF₂CHFCF₂CF₂—, —CH₂CF₂CHFCF₂CF₂—, —CH₂CF₂CF₂CF₂CH₂—, and -cycloC₅F₈—.

Specific examples of R^{f6} include —CF₂CF₂CF₂CF₂CF₂CF₂—, —CF₂CF₂CHFCHFCF₂CF₂—, —CHFCF₂CF₂CF₂CF₂CF₂—, —CHFCHFCHFCHFCHFCHF—, —CHFCF₂CF₂CF₂CF₂CH₂—, —CH₂CF₂CF₂CF₂CF₂CH₂—, and -cycloC₆F₁₀—.

Here, -cycloC₄F₆— means a perfluorocyclobutanediyl group, and specific examples thereof include a perfluorocyclobutane-1,2-diyl group. -cycloC₅F₈— means a perfluorocyclopentanediyl group, and specific examples thereof include a perfluorocyclopentane-1,3-diyl group. -cycloC₆F₁₀— means a perfluorocyclohexanediyl group, and specific examples thereof include a perfluorocyclohexane-1,4-diyl group.

Among them, R^{f} preferably has a structure represented by the following formulae (F2) to (F4) from the viewpoint of further excellent water/oil repellency, friction resistance, and fingerprint dirt removability.

(R^{f1}O)ₘ₁—(R^{f2}O)ₘ₂—(R^{f7})ₘ₇ Formula (F2)

(R^{f2}O)ₘ₂—(R^{f4}O)ₘ₄—(R^{f7})ₘ₇ Formula (F3)

(R^{f3}O)ₘ₃—(R^{f7})ₘ₇ Formula (F4)
where, each reference sign of the formulae (F2) to (F4) is the same as those in the formula (F1).

In the formulae (F2) and (F3), the bonding order of (R^{f1}O) and (R^{f2}O), and (R^{f2}O) and (R^{f4}O) is random. For example, (R^{f1}O) and (R^{f2}O) may be alternately arranged, (R^{f1}O) and (R^{f2}O) may be each arranged in a block, or may be random. The same applies to the formula (F3).

In the formula (F2), m1 is preferably 1 to 30, and more preferably 1 to 20. In addition, m2 is preferably 1 to 30, and more preferably 1 to 20.

In the formula (F3), m2 is preferably 1 to 30, and more preferably 1 to 20. In addition, m4 is preferably 1 to 30, and more preferably 1 to 20.

In the formula (F4), m3 is preferably 1 to 30, and more preferably 1 to 20. The ratio of fluorine atoms in the polyfluoropolyether chain R^{f} [{number of fluorine atoms/(number of fluorine atoms+number of hydrogen atoms)}×100(%)] is preferably 40% or more, more preferably 50% or more, and still more preferably 60% or more from the viewpoint of excellent water/oil repellency and fingerprint removability.

In addition, the molecular weight of the R^{f} portion of the polyfluoropolyether chain is preferably 200 to 30,000, more preferably 600 to and still more preferably 1,000 to 20,000 from the viewpoint of wear resistance.

In the compound (A), T is Si(—R⁶)₃₋ₐ(—R⁷)ₐ, R⁶ is a hydrogen atom or a hydrocarbon group, and R⁷ is a hydrolyzable group or a hydroxyl group.

When R⁷ is a hydroxyl group, a silanol (Si—OH) group is constituted together with a Si atom. In addition, the hydrolyzable group is a group that becomes a hydroxyl group by a hydrolysis reaction. The silanol group further reacts between molecules to form a Si—O—Si bond. In addition, the silanol group undergoes a dehydration condensation reaction with a hydroxyl group (substrate —OH) on the surface of the substrate to form a chemical bond (substrate —O—Si). The present compound (A) having one or more T's is excellent in wear resistance after surface layer formation.

Examples of the hydrolyzable group include an alkoxy group, an aryloxy group, a halogen atom, an acyl group, an acyloxy group, and an isocyanate group (—NCO). The alkoxy group is preferably an alkoxy group having 1 to 4 carbon atoms. The acyl group is preferably an acyl group having 1 to 6 carbon atoms. The acyloxy group is preferably an acyloxy group having 1 to 6 carbon atoms.

Among them, R⁷ is preferably an alkoxy group having 1 to 4 carbon atoms or a halogen atom from the viewpoint of ease of production of the present compound. The alkoxy group in R⁷ is preferably an alkoxy group having 1 to 4 carbon atoms from the viewpoint of being excellent in storage stability of the present compound and suppressing outgassing during the reaction, particularly preferably an ethoxy group from the viewpoint of long-term storage stability, and particularly preferably a methoxy group from the viewpoint of shortening the hydrolysis reaction time. In addition, the halogen atom is preferably a chlorine atom.

R⁶ is a hydrogen atom or a monovalent hydrocarbon group. Examples of the hydrocarbon group include an alkyl group, a cycloalkyl group, an alkenyl group, and an allyl group, and an alkyl group is preferable from the viewpoint of ease of production and the like. In addition, from the viewpoint of ease of production and the like, the number of carbon atoms in the hydrocarbon group is preferably 1 to 6, more preferably 1 to 3, and still more preferably 1 and 2.

The number a of R⁷ in one T may be 1 to 3, preferably 2 or 3, and more preferably 3 from the viewpoint of adhesion to the substrate.

Specific examples of T include —Si(OCH₃)₃, —SiCH₃(OCH₃)₂, —Si(OCH₂CH₃)₃, —SiCl₃, —Si(OCOCH₃)₃, and —Si(NCO)₃. From the viewpoint of ease of handling in production, —Si(OCH₃)₃ is particularly preferable.

The number n2 of T in one molecule of the compound (A) may be 1 to 20, and n2 is preferably 1 to 12 and more preferably 1 to 6 from the viewpoint of ease of synthesis, ease of handling of the compound (A), and the like.

When there are two or more T's in one molecule of the compound (A), the T may have the same structure as or different structures from each other.

Q¹ is an (n1+1)-valent linking group that links R¹R²C═CR³-L¹ and R^{f}, and Q² is an (n2+1)-valent linking group that links T and R^{f}. Q¹ and Q² in the compound (A) may have the same structure as or different structures from each other. Since the specific structures of Q¹ and Q² are common, Q¹ will be described below as a representative, and Q² conforms to Q¹ unless otherwise specified.

When Q¹ is a trivalent or higher valent linking group, at least one branch point (hereinafter referred to as “branch point P¹”) selected from the group consisting of C, N, Si, a ring structure, and a (n1+1)-valent organopolysiloxane residue is preferably provided.

The ring structure constituting the branch point P¹ is preferably one selected from the group consisting of a 3 to 8 membered aliphatic ring, a 3 to 8 membered aromatic ring, a 3 to 8 membered heterocyclic ring, and a condensed ring consisting of two or more of these rings, and particularly preferably a ring structure represented by the following formula from the viewpoint of easy production of the present compound and further excellent friction resistance, light fastness, and chemical resistance of the surface layer. The ring structure may have a substituent such as a halogen atom, an alkyl group (an etheric oxygen atom may be contained between the carbon-carbon atoms), a cycloalkyl group, an alkenyl group, an allyl group, an alkoxy group, or an oxo group (═O).

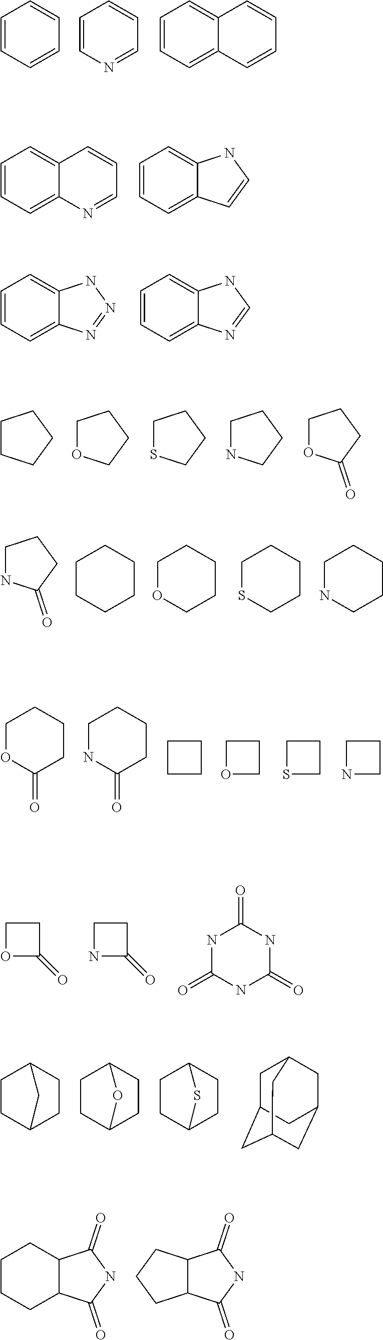

Examples of the organopolysiloxane residue constituting the branch point P¹ include the following groups. Here, R²⁵ in the following formula is a hydrogen atom, an alkyl group, an alkoxy group, or a phenyl group. The number of carbon atoms in the alkyl group and the alkoxy group of R²⁵ is preferably 1 to 10, and more preferably 1.

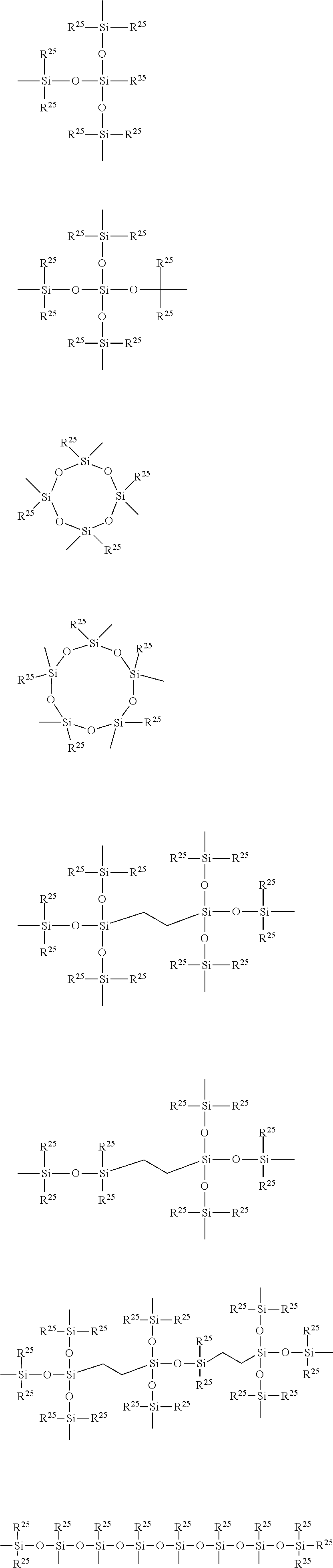

The divalent or higher valent Q¹ may have at least one bond (hereinafter referred to as “bond B¹”) selected from the group consisting of —C(O)NR²⁶—, —C(O)O—, —C(O)—, —O—, —NR²⁶—, —S—, —OC(O)O—, —NHC(O)O—, —NHC(O)NR²⁶—, —SO₂NR²⁶—, —Si(R²⁶)₂—, —OSi(R²⁶)₂—, —Si(CH₃)₂-Ph-Si(CH₃)₂—, and a divalent organopolysiloxane residue. Here, R²⁶ is a hydrogen atom, an alkyl group having 1 to 6 carbon atoms, or a phenyl group, and Ph is a phenylene group. The number of carbon atoms in the alkyl group of R²⁶ is preferably 1 to 3 and more preferably 1 and 2 from the viewpoint of easy production of the present compound.

Examples of the divalent organopolysiloxane residue include the following groups. Here, R²⁷ in the following formula is a hydrogen atom, an alkyl group, an alkoxy group, or a phenyl group. The number of carbon atoms in the alkyl group and the alkoxy group of R²⁷ is preferably 1 to 10, and more preferably 1.

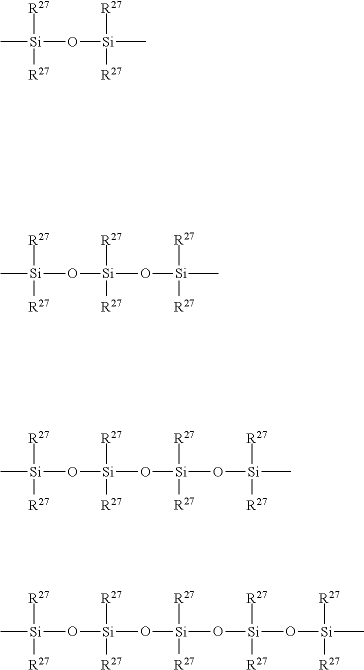

The bond B¹ is preferably at least one bond selected from the group consisting of —C(O)NR²⁶—, —C(O)—, —NR²⁶—, and —O— from the viewpoint of easy production of the present compound, and is more preferably C(O)NR²⁶— or —C(O)— from the viewpoint of further excellent light fastness and chemical resistance of the surface layer.

Examples of the trivalent or higher Q¹ include a combination of two or more divalent hydrocarbon groups R²⁸ and one or more branch points P¹ (for example, {P¹—R²⁸—)ₙ₁₊₁}), and a combination of two or more hydrocarbon groups R²⁸, one or more branch points P¹, and one or more bonds B¹ (for example, {P¹—(B¹—R²⁸—)ₙ₁₊₁}).

In addition, examples of the divalent Q¹ include a single bond, a divalent hydrocarbon group R²⁸, and a combination of one or two divalent hydrocarbon groups R²⁸ and a bond B¹ (for example, R²⁸—B¹—, or —B¹—R²⁸—B¹—).

Examples of the divalent hydrocarbon group include a divalent aliphatic hydrocarbon group (alkylene group, cycloalkylene group, and the like) and a divalent aromatic hydrocarbon group (phenylene group or the like). The number of carbon atoms in the divalent hydrocarbon group is preferably 1 to 10, more preferably 1 to 6, and still more preferably 1 to 4.

The Q¹ is preferably a group represented by any one of the following formulae (Q1) to (Q7) from the viewpoint of easy production of the present compound.

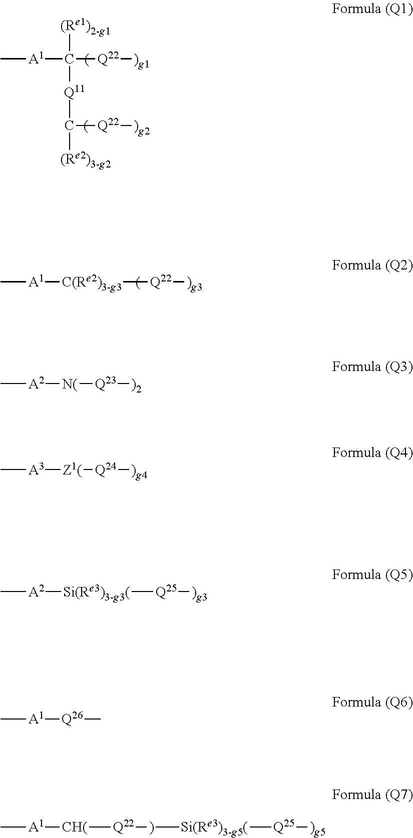

where, in the formulae (Q1) to (Q7), the A¹, A², or A³ side is connected to R^{f} of the general formula (A), and the Q²², Q²³, Q²⁴, Q²⁵, or Q²⁶ side is connected to R¹R²C═CR³-L¹ or T,
A¹ is a single bond, an alkylene group, or a group having C(O)NR^{e6}—, —C(O)—, —NR^{e6}—, or —O— between carbon-carbon atoms of an alkylene group having 2 or more carbon atoms,
A² is an alkylene group or a group having —C(O)NR^{e6}—, —C(O)—, —NR^{e6}—, or —O— between carbon-carbon atoms of an alkylene group having 2 or more carbon atoms,
A³ is A¹ when the atom in Z¹ to which A³ is bonded is a carbon atom, and A² when the atom in Z¹ to which A³ is bonded is a nitrogen atom,
Q¹¹ is a single bond, —O—, an alkylene group, or a group having C(O)NR^{e6}—, —C(O)—, —NR^{e6}—, or —O— between carbon-carbon atoms of an alkylene group having 2 or more carbon atoms,
Q²² is an alkylene group, a group having —C(O)NR^{e6}—, —C(O)—, —NR^{e6}—, or —O— between carbon-carbon atoms of an alkylene group having 2 or more carbon atoms, a group having —C(O)NR^{e6}—, —C(O)—, —NR^{e6}—, or —O— at the terminal of the alkylene group on the side not connected to R¹R²C═CR³-L¹ or T, or a group having —C(O)NR^{e6}—, —C(O)—, —NR^{e6}—, or —O— between carbon-carbon atoms of an alkylene group having 2 or more carbon atoms, and —C(O)NR^{e6}—, —C(O)—, —NR^{e6}—, or —O— at the terminal on the side not connected to R¹R²C═CR³-L¹ or T, and when Q¹ has two or more Q²²'s, Q²²'s may the two or more be the same as or different from each other,
Q²³ is an alkylene group or a group having —C(O)NR^{e6}—, —C(O)—, —NR^{e6}—, or —O— between carbon-carbon atoms of an alkylene group having 2 or more carbon atoms, and two Q²³'s may be the same or different;
Q²⁴ is Q²² when the atom in Z¹ to which Q²⁴ is bonded is a carbon atom, and Q²³ when the atom in Z¹ to which Q²⁴ is bonded is a nitrogen atom, and when Q¹ has two or more Q²⁴'s, Q²⁴'s may the two or more Q²⁴'s may be the same as or different from each other,
Q²⁵ is an alkylene group or a group having —C(O)NR^{e6}—, —C(O)—, —NR^{e6}—, or —O— between carbon-carbon atoms of an alkylene group having 2 or more carbon atoms, and when Q¹ has two or more Q²⁵'s, the two or more Q²⁵'s may be the same as or different from each other,
Q²⁶ is an alkylene group or a group having —C(O)NR^{e6}—, —C(O)—, —NR^{e6}—, or —O— between carbon-carbon atoms of an alkylene group having 2 or more carbon atoms,
Z¹ is a group having a (g+1)-valent ring structure which has a carbon atom or a nitrogen atom to which A³ is directly bonded and has a carbon atom or a nitrogen atom to which Q²⁴ is directly bonded,
R^{e1} is a hydrogen atom or an alkyl group, and when Q¹ has two or more R^{e1}'s, the two or more R^{e1}'s may be the same as or different from each other,
R^{e2} is a hydrogen atom, a hydroxyl group, an alkyl group, or an acyloxy group,
R^{e3} a is an alkyl group,
R^{e6} is a hydrogen atom, an alkyl group having 1 to 6 carbon atoms, or a phenyl group,
g1 is an integer of 0 to 3, g2 is an integer of 0 to 3, and g1+g2 is an integer of 1 to 6,
g3 is an integer of 1 to 3.
g4 is an integer of 1 or more, and
g5 is an integer of 0 to 3.

Note that g1+g2=g, g3=g, g4=g, and g5+1=g.

The number of carbon atoms in the alkylene group of Q¹¹, Q²², Q²³, Q²⁴, Q²⁵, or Q²⁶ is preferably 1 to 10, more preferably 1 to 6, and still more preferably 1 to 4 from the viewpoint of easy production of the present compound and further excellent friction resistance, light fastness, and chemical resistance of the surface layer. However, the lower limit value of the number of carbon atoms of the alkylene group in the case of having a specific bond between carbon-carbon atoms is 2.

Examples of the ring structure in Z¹ include the ring structures described above, and preferred forms are also the same. In addition, since Q²⁴ is directly bonded to the ring structure in Z¹, for example, an alkylene group is linked to the ring structure and Q²⁴ is not linked to the alkylene group.

The number of carbon atoms in the alkyl group of R^{e1}, R^{e2}, or R^{e3} is preferably 1 to 6, more preferably 1 to 3, and still more preferably 1 and 2 from the viewpoint of easy production of the present compound.

The number of carbon atoms in the alkyl group part of the acyloxy group of R^{e2} is preferably 1 to 6, more preferably 1 to 3, and still more preferably 1 and 2 from the viewpoint of easy production of a compound 1.

g4 is preferably 2 to 6, more preferably 2 to 4, and still more preferably 2 or 3 from the viewpoint of easy production of the present compound and further excellent friction resistance and fingerprint dirt removability of the surface layer.

Another form of Q¹ includes a group represented by any one of the following formulae (Q11) to (Q17).

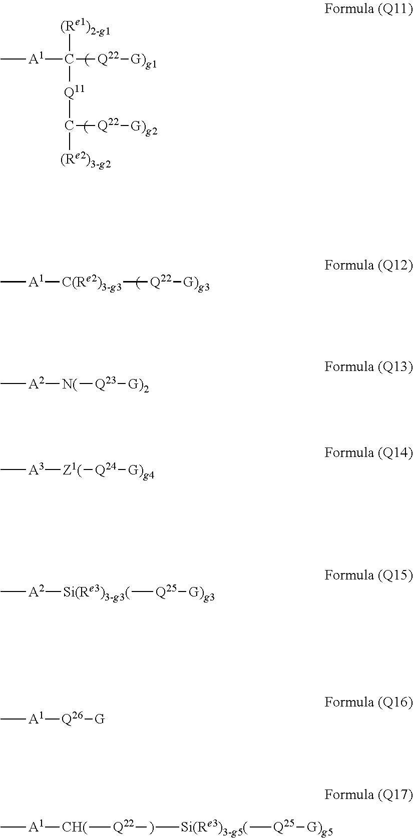

However, in the formulae (Q11) to (Q17), the A¹, A², or A³ side is connected to R^{f} of the general formula (A), and the Q²², Q²³, Q²⁴, Q² or Q²⁶ side is connected to R¹R²C═CR³-L¹ or T. G is the following group g3, and two or more Gs of Q¹ may be the same as or different from each other. The reference signs other than G are the same as the signs in formulae (Q1) to (Q7).

—Si(R²¹)₃₋ₖ(-Q³-)ₖ Formula g3

However, in formula g3, the Si side is connected to Q²², Q²³, Q²⁴, Q²⁵, or Q²⁶, and the Q³ side is connected to R¹R²C═CR³-L¹ or T. R²¹ is an alkyl group. Q³ is an alkylene group, a group having —C(O)NR²⁶—, —C(O)—, —NR²⁶—, or —O— between carbon-carbon atoms of an alkylene group having 2 or more carbon atoms, or —(OSi(R²²)₂)ₚ—O—, and two or more Q³'s may be the same as or different from each other. k is 2 or 3. R²⁶ is a hydrogen atom, an alkyl group having 1 to 6 carbon atoms, or a phenyl group. R²² is an alkyl group, a phenyl group, or an alkoxy group, and two R²²'s may be the same as or different from each other. p is an integer of 0 to 5, and when p is 2 or more, two or more (OSi(R²²)₂)'s may be the same as or different from each other.

The number of carbon atoms in the alkylene group of Q³ is preferably 1 to more preferably 1 to 6, and still more preferably 1 to 4 from the viewpoint of easy production of the present compound and further excellent friction resistance, light fastness, and chemical resistance of the surface layer. However, the lower limit value of the number of carbon atoms of the alkylene group in the case of having a specific bond between carbon-carbon atoms is 2.

The number of carbon atoms in the alkyl group of R²¹ is preferably 1 to 6, more preferably 1 to 3, and still more preferably 1 and 2 from the viewpoint of easy production of the present compound.

The number of carbon atoms in the alkyl group of R²² is preferably 1 to 6, more preferably 1 to 3, and still more preferably 1 and 2 from the viewpoint of easy production of the present compound.

The number of carbon atoms in the alkoxy group of R²² is preferably 1 to 6, more preferably 1 to 3, and still more preferably 1 and 2 from the viewpoint of excellent storage stability of the present compound.

p is preferably 0 or 1.

The plurality of the present compounds may be one single compound corresponding to the formula (A) or a mixture of two or more compounds corresponding to the formula (A).

The molecular weight of the present compound is preferably 500 to 100,000, and particularly preferably 1,000 to 20,000. In addition, when the present compound consists of a mixture of two or more compounds, the molecular weight distribution (Mw/Mn) of the compounds is preferably 1.0 to 2.0, and particularly preferably 1.0 to 1.3. When the molecular weight and the molecular weight distribution are within the above ranges, there are advantages that the viscosity is low, the evaporation component is small, and the uniformity when dissolved in a solvent is excellent. The molecular weight and molecular weight distribution of the present compound can be measured by gel permeation chromatography, and as the measurement conditions, the conditions described in Examples, which will be described later, can be adopted.

Specific examples of the present compound include the following compounds. The compound of the following formula is preferable from the viewpoint of industrially easy production, ease of handling, and the further excellent water/oil repellency, friction resistance, fingerprint dirt removability, lubricity, chemical resistance, light fastness, and chemical resistance of the surface layer, and among these, particularly, excellent light fastness.

CH₂═CF—O—R^{f}—C(═O)—NH—CH₂—C(CH₂CH₂CH₂-T)₃,

CH₂═CF—O—R^{f}—C(═O)—N(CH₂CH₂CH₂-T)₂,

CH₂═CF—O—R^{f}—C(═O)—NH—CH₂CH₂CH₂-T,

CH₂═CF—O—R^{f}—C(OH)(—CH₂CH₂CH₂-T)₂,

CH₂═CF—O—R^{f}—C(OR³⁰)(—CH₂CH₂CH₂-T)₂,

CH₂═CF—O—R^{f}—CH₂—CHCH₂-Q³⁰(—CH₂CH₂-T)ₙ₃,

CH₂═CF—CF₂—R^{f}—C(═O)—NH—CH₂—C(CH₂CH₂CH₂-T)₃,

CH₂═CF—CF₂—R^{f}—C(═O)—N(CH₂CH₂CH₂-T)₂,

CH₂═CF—CF₂—R^{f}—C(═O)—NH—CH₂CH₂CH₂-T,

CH₂═CF—CF₂—R^{f}—C(OH)(—CH₂CH₂CH₂-T)₂,

CH₂═CF—CF₂—R^{f}—C(OR³⁰)(—CH₂CH₂CH₂-T)₂,

CH₂═CF—CF₂—R^{f}—CH₂—CHCH₂-Q³⁰(—CH₂CH₂-T)ₙ₃,

CH₂═CF—CF₂CF₂—R^{f}—C(═O)—NH—CH₂—C(CH₂CH₂CH₂-T)₃,

CH₂═CF—CF₂CF₂—R^{f}—C(═O)—N(CH₂CH₂CH₂-T)₂,

CH₂═CF—CF₂CF₂—R^{f}—C(═O)—NH—CH₂CH₂CH₂-T,

CH₂═CF—CF₂CF₂—R^{f}—C(OH)(—CH₂CH₂CH₂-T)₂,

CH₂═CF—CF₂CF₂—R^{f}—C(OR³⁰)(—CH₂CH₂CH₂-T)₂,

CH₂═CF—CF₂CF₂—R^{f}—CH₂—CHCH₂-Q³⁰(—CH₂CH₂-T)ₙ₃,

CH₂═C(CF₃)—O—R^{f}—C(═O)—NH—CH₂—C(CH₂CH₂CH₂-T)₃,

CH₂═C(CF₃)—O—R^{f}—C(═O)—N(CH₂CH₂CH₂-T)₂,

CH₂═C(CF₃)—O—R^{f}—C(═O)—NH—CH₂CH₂CH₂-T,

CH₂═C(CF₃)—O—R^{f}—C(OH)(—CH₂CH₂CH₂-T)₂,

CH₂═C(CF₃)—O—R^{f}—C(OR³⁰)(—CH₂CH₂CH₂-T)₂,

CH₂═C(CF₃)—O—R^{f}—CH₂—CHCH₂-Q³⁰(—CH₂CH₂-T)ₙ₃,

CH₂═C(CF₃)—CF₂—R^{f}—C(═O)—NH—CH₂—C(CH₂CH₂CH₂-T)₃,

CH₂═C(CF₃)—CF₂—R^{f}—C(═O)—N(CH₂CH₂CH₂-T)₂,

CH₂═C(CF₃)—CF₂—R^{f}—C(═O)—NH—CH₂CH₂CH₂-T,

CH₂═C(CF₃)—CF₂—R^{f}—C(OH)—(CH₂CH₂CH₂-T)₂,

CH₂═C(CF₃)—CF₂—R^{f}—C(OR³⁰)—(CH₂CH₂CH₂-T)₂,

CH₂═C(CF₃)—CF₂—R^{f}—CH₂—CHCH₂-Q³⁰(—CH₂CH₂-T)ₙ₃,

CH₂═C(CF₃)—CF₂CF₂—R^{f}—C(═O)—NH—CH₂—C(CH₂CH₂CH₂-T)₃,

CH₂═C(CF₃)—CF₂CF₂—R^{f}—C(═O)—N(CH₂CH₂CH₂-T)₂,

CH₂═C(CF₃)—CF₂CF₂—R^{f}—C(═O)—NH—CH₂CH₂CH₂-T,

CH₂═C(CF₃)—CF₂CF₂—R^{f}—C(OH)—(CH₂CH₂CH₂-T)₂,

CH₂═C(CF₃)—CF₂CF₂—R^{f}—C(OR³⁰)—(CH₂CH₂CH₂-T)₂,

CH₂═C(CF₃)—CF₂CF₂—R^{f}—CH₂—CHCH₂-Q³⁰(—CH₂CH₂-T)ₙ₃,
where, in the formula, R^{f} and T are as described above, R³⁰ is an alkyl group or a fluoroalkyl group, Q³⁰ is a (1+n3)-valent linking group, and n3 is an integer of 1 to 20. Examples of Q³⁰ include those similar to Q² described above.

### (Method for Producing Present Compound)

A method for producing the present compound will be described with an example. The production method is not limited to the following method, but according to the following method, the present compound can be obtained with high yield.

The compound (A) as the present compound can be produced, for example, by hydrosilylation reaction of the following compound (B) and the following compound (C1) or compound (C2).

(R¹R²C═CR³-L¹-)ₙ₁Q¹-R^{f}-Q¹²(-L¹¹-CR¹³═CR¹¹R¹²)ₙ₂ Formula (B)

HSi(—R⁶)₃₋ₐ(—R⁷)ₐ Formula (C1)

HSi(R⁴¹)₃₋ₖ[—(OSi(R⁴²)₂)ₚ₁—O—Si(—R⁶)₃₋ₐ(—R⁷)ₐ]ₖ Formula (C2)
where
L¹¹ is an oxygen atom or CR¹⁴R¹⁵, and when there are a plurality of L¹¹'s, the plurality of L¹¹'s may be the same as or different from each other,
R¹¹ and R¹² are each independently a hydrogen atom or an alkyl group which may have a substituent, and when there are a plurality of R¹¹'s and R¹²'s, the plurality of R¹¹'s and R¹²'s may be the same as or different from each other,
R¹³ is a hydrogen atom, a halogen atom, or an alkyl group which may have a substituent, and when there are a plurality of R¹³'s, the plurality of R¹³'s may be the same as or different from each other,
R¹⁴ and R¹⁵ are each independently a hydrogen atom, a halogen atom, or an alkyl group which may have a substituent, and when there are a plurality of R¹⁴'s and R¹⁵'s, the plurality of R¹⁴'s and R¹⁵'s may be the same as or different from each other,
Q¹² is an (n2+1)-valent linking group,
R⁴¹ is an alkyl group, and when there are a plurality of R⁴¹'s, the plurality of R⁴¹'s may be the same as or different from each other,
R⁴² is an alkyl group, a phenyl group, or an alkoxy group, a plurality of R⁴²'s may be the same as or different from each other,
k is 2 or 3,
p1 is an integer of 0 to 5, and when p1 is 2 or more, two or more (OSi(R⁴²)₂) may be the same as or different from each other,
R¹, R², R³, R⁴, R⁵, R⁶, R⁷, Q¹, R^{f}, n1, n2, and a are the same as those in the compound (A), and preferred embodiment is also the same, and
R¹R²C═CR³-L¹- and R¹¹R¹²C═CR¹³-L¹¹- are different structures.

In addition, the compound (C2) can be produced, for example, by the method described in International Patent Publication No. WO 2019/208503.

The alkyl group which may have a substituent and the halogen atom in R¹¹, R¹², R¹³, R¹⁴, and R¹⁵ are the same as those described in R¹ to R⁵ of the compound (A), and preferred embodiments are also the same.

In addition, after the above reaction, Q¹²(-L¹¹R¹³CH—CR¹²R¹¹)ₙ₂ corresponds to the linking group Q² in the compound (A).

The synthesis method is a technique in which the compound (B) in which R¹R²C═CR³-L¹ and R¹¹R¹²C═CR¹³L¹¹ have structures different from each other is used, and a difference in reactivity between R¹R²C═CR³-L¹ and R L¹¹ is utilized, and accordingly, a reaction of one of olefins of the compound (B) is allowed to proceed dominantly, thereby increasing the yield of the compound (A).

For example, when R is an alkyl group, CH₂═CH— has higher reactivity than RCH═CH—. Similarly, when X is a halogen atom, CH₂═CH— has higher reactivity than CH₂═CX—. In addition, CH₂═CH—CH₂— has higher reactivity than CH₂═CH—CF₂—.

Specific examples of the compound (B) include the following compounds.

CH₂═CF—O—R^{f}—C(═O)—NH—CH₂—C(CH₂—CH═CH₂)₃,

CH₂═CF—O—R^{f}—C(═O)—N(CH₂—CH═CH₂)₂,

CH₂═CF—O—R^{f}—C(═O)—NH—CH₂—CH═CH₂,

CH₂═CF—O—R^{f}—C(OH)(—CH₂—CH═CH₂)₂,

CH₂═CF—O—R^{f}—C(OR³⁰)(—CH₂—CH═CH₂)₂,

CH₂═CF—O—R^{f}—CH₂—CHCH₂-Q³²(—CH₂—CH═CH₂)ₙ₃,

CH₂═CF—CF₂—R^{f}—C(═O)—NH—CH₂—C(CH₂—CH═CH₂)₃,

CH₂═CF—CF₂—R^{f}—C(═O)—N(CH₂—CH═CH₂)₂,

CH₂═CF—CF₂—R^{f}—C(═O)—NH—CH₂—CH═CH₂T,

CH₂═CF—CF₂—R^{f}—C(OH)(—CH₂—CH═CH₂)₂,

CH₂═CF—CF₂—R^{f}—C(OR³⁰)(—CH₂—CH═CH₂)₂,

CH₂═CF—CF₂—R^{f}—CH₂—CHCH₂-Q³²(—CH₂—CH═CH₂)ₙ₃,

CH₂═CF—CF₂CF₂—R^{f}—C(═O)—NH—CH₂—C(CH₂—CH═CH₂)₃,

CH₂═CF—CF₂CF₂—R^{f}—C(═O)—N(CH₂—CH═CH₂)₂,

CH₂═CF—CF₂CF₂—R^{f}—C(═O)—NH—CH₂—CH═CH₂,

CH₂═CF—CF₂CF₂—R^{f}—C(OH)(—CH₂—CH═CH₂)₂,

CH₂═CF—CF₂CF₂—R^{f}—C(OR³⁰)(—CH₂—CH═CH₂)₂,

CH₂═CF—CF₂CF₂—R^{f}—CH₂—CHCH₂-Q³²(—CH₂—CH═CH₂)ₙ₃,

CH₂═C(CF₃)—O—R^{f}—C(═O)—NH—CH₂—C(CH₂—CH═CH₂)₃,

CH₂═C(CF₃)—O—R^{f}—C(═O)—N(CH₂—CH═CH₂)₂,

CH₂═C(CF₃)—O—R^{f}—C(═O)—NH—CH₂—CH═CH₂,

CH₂═C(CF₃)—O—R^{f}—C(OH)(—CH₂—CH═CH₂)₂,

CH₂═C(CF₃)—O—R^{f}—C(OR³⁰)(—CH₂—CH═CH₂)₂,

CH₂═C(CF₃)—O—R^{f}—CH₂—CHCH₂-Q³²(—CH₂—CH═CH₂)ₙ₃,

CH₂═C(CF₃)—CF₂—R^{f}—C(═O)—NH—CH₂—C(CH₂—CH═CH₂)₃,

CH₂═C(CF₃)—CF₂—R^{f}—C(═O)—N(CH₂—CH═CH₂)₂,

CH₂═C(CF₃)—CF₂—R^{f}—C(═O)—NH—CH₂—CH═CH₂,

CH₂═C(CF₃)—CF₂—R^{f}—C(OH)—(CH₂—CH═CH₂)₂,

CH₂═C(CF₃)—CF₂—R^{f}—C(OR³⁰)—(CH₂—CH═CH₂)₂,

CH₂═C(CF₃)—CF₂—R^{f}—CH₂—CHCH₂-Q³²(—CH₂—CH═CH₂)ₙ₃,

CH₂═C(CF₃)—CF₂CF₂—R^{f}—C(═O)—NH—CH₂—C(CH₂—CH═CH₂)₃,

CH₂═C(CF₃)—CF₂CF₂—R^{f}—C(═O)—N(CCH₂—CH═CH₂)₂,

CH₂═C(CF₃)—CF₂CF₂—R^{f}—C(═O)—NH—CH₂—CH═CH₂,

CH₂═C(CF₃)—CF₂CF₂—R^{f}—C(OH)—(CH₂—CH═CH₂)₂,

CH₂═C(CF₃)—CF₂CF₂—R^{f}—C(OR³⁰)—(CH₂—CH═CH₂)₂,

CH₂═C(CF₃)—CF₂CF₂—R^{f}—CH₂—CHCH₂-Q³²(—CH₂—CH═CH₂)ₙ₃,
where, R^{f}, T, R³⁰, and n3 are as described above, and Q³² is an (n3+1)-valent linking group, and is the same as Q¹².

In addition, in the above production method, a compound represented by the following formula (D) or the like may be produced as a by-product. In this case, depending on the application of the compound (B) and the like, the compound (B) and the compound (D) may be separated by a known column chromatography method or the like, or a mixture of the compound (B) and the compound (D) may be used as it is.

(T-)ₙ₁Q¹-R^{f}-Q²(-T)ₙ₂ Formula (D)
where, each reference sign in the formula is the same as those of the compound (A).

Examples of the method for producing the compound (B) include a method in which the following compound (F) is reacted with the following compound (E), the compound (G) is obtained by column chromatography or the like as necessary, and then a highly reactive olefin is introduced into the ester of the compound (G).

H₃CO—C(O)-Q¹-R^{f}-Q³¹-(C(O)OCH₃)ₙ₄ Formula (E)

H₂NCH₂C(CH₂CH═CH₂)₃ Formula (F)

H₃CO—C(O)-Q¹-R^{f}-Q³¹-(C(O)—NH—CH₂—C(CH₂—CH═CH₂)₃)ₙ₄ Formula (G)
where Q¹ and R^{f} in the formula are as described above, Q³¹ is a (1+n4)-valent linking group, and n4 is an integer of 1 to 20.

### [Fluorine-Containing Ether Composition]

The fluorine-containing ether composition of the present invention (hereinafter also referred to as the present composition) is a composition containing the compound (A), and at least one of a fluorine-containing compound other than the present compound and the following impurities. Examples of the impurities include the compound (A) and compounds (for example, the compound (B) and the compound (D)) unavoidable in the production of other fluorine-containing compounds. In addition, the present composition does not contain a liquid medium which will be described later.

Examples of other fluorine-containing compounds include a fluorine-containing compound by-produced in the production process of the present compound (hereinafter also referred to as by-product fluorine-containing compound), and a known fluorine-containing compound used for the same application as the present compound.

As other fluorine-containing compounds, a compound that is less likely to deteriorate the properties of the present compound is preferable.

The content of other fluorine-containing compounds is preferably less than 50% by mass, more preferably less than 30% by mass, and still more preferably less than 10% by mass in the total amount of the present composition from the viewpoint of sufficiently exhibiting the characteristics of the present compound.

Examples of the by-product fluorine-containing compound include unreacted fluorine-containing compounds at the time of synthesis of the present compound. When the present composition contains a by-product fluorine-containing compound, a purification process for removing the by-product fluorine-containing compound or reducing the amount of the by-product fluorine-containing compound can be simplified.

Examples of the known fluorine-containing compound include those described in the following documents.
perfluoropolyether-modified aminosilane described in Japanese Unexamined Patent Application Publication No. 11-029585,
a silicon-containing organic fluorine-containing polymer described in Japanese Patent No. 2874715,
an organosilicon compound described in Japanese Unexamined Patent Application Publication No. 2000-144097;
perfluoropolyether-modified aminosilane described in Japanese Unexamined Patent Application Publication No. 2000-327772,
a fluorinated siloxane described in Published Japanese Translation of PCT International Publication for Patent Application, No. 2002-506887,
an organic silicone compound described in Published Japanese Translation of PCT International Publication for Patent Application, No. 2008-534696,
a fluorinated modified hydrogen-containing polymer described in Japanese Patent No. 4138936,
compounds described in United States Patent Publication No. 2010/0129672, International Patent Publication No. WO 2014/126064, and Japanese Unexamined Patent Application Publication No. 2014-070163,
organosilicon compounds described in International Patent Publication No. WO 2011/060047 and International Patent Publication No. WO 2011/059430,
a fluorine-containing organosilane compound described in International Patent Publication No. WO 2012/064649,
a fluorooxyalkylene group-containing polymer described in Japanese Unexamined Patent Application Publication No. 2012-72272,
fluorine-containing ether compounds described in International Patent Publication No. WO 2013/042732, International Patent Publication No. WO 2013/121984, International Patent Publication No. WO 2013/121985, International Patent Publication No. WO 2013/121986, International Patent Publication No. WO 2014/163004, Japanese Unexamined Patent Application Publication No. 2014-080473, International Patent Publication No. WO 2015/087902, International Patent Publication No. WO 2017/038830, International Patent Publication No. WO 2017/038832, and International Patent Publication No. WO 2017/187775,
perfluoro(poly)ether-containing silane compounds described in Japanese Unexamined Patent Application Publication No. 2014-218639, International Patent Publication No. WO 2017/022437, International Patent Publication No. WO 2018/079743, and International Patent Publication No. WO 2018/143433,
fluoropolyether group-containing polymer-modified silane described in Japanese Unexamined Patent Application Publication No. 2015-199906, Japanese Unexamined Patent Application Publication No. 2016-204656, Japanese Unexamined Patent Application Publication No. 2016-210854, and Japanese Unexamined Patent Application Publication No. 2016-222859, and
fluorine-containing ether compounds described in International Patent Publication No. WO 2018/216630, International Patent Publication No. WO 2019/039226, International Patent Publication No. WO 2019/039341, International Patent Publication No. WO 2019/039186, International Patent Publication No. WO 2019/044479, Japanese Unexamined Patent Application Publication No. 2019-44158, and International Patent Publication No. WO 2019/163282.

In addition, examples of commercially available products of the fluorine-containing compound include KY-100 series (KY-178, KY-185, KY-195, and the like) manufactured by Shin-Etsu Chemical Co., Ltd., Afluid (registered trademark) 5550 manufactured by AGC Inc., and OPTOOL (registered trademark) DSX, OPTOOL (registered trademark) AES, OPTOOL (registered trademark) UF503, and OPTOOL (registered trademark) UD509 manufactured by manufactured by Daikin Industries, Ltd.

The ratio of the present compound in the present composition is less than 100% by mass, preferably 60% by mass or more, more preferably 70% by mass or more, and still more preferably 80% by mass or more.

When the present composition contains other fluorine-containing compounds, the ratio of other fluorine-containing compounds to the total of the present compound and other fluorine-containing compounds in the present composition is preferably 40% by mass or less, more preferably 30% by mass or less, and still more preferably 20% by mass or less.

The total ratio of the present compound and other fluorine-containing compounds in the present composition is preferably 80% by mass or more, and more preferably 85% by mass or more.

When the content of the present compound and other fluorine-containing compounds is within the above range, the surface layer has excellent water/oil repellency, friction resistance, fingerprint dirt removability, lubricity, and appearance.

### [Coating Liquid]

The coating liquid of the present invention (hereinafter also referred to as the present coating liquid) contains the present compound or the present composition and a liquid medium. The present coating liquid may be a liquid, may be a solution, or may be a dispersion.

The present coating liquid may contain the present compound or the present composition, and may contain impurities such as by-products generated in the production process of the present compound.

The concentration of the present compound or the present composition is preferably 0.001 to 40% by mass, more preferably 0.01 to 20% by mass, and still more preferably 0.1 to 10% by mass in the present coating liquid.

The liquid medium is preferably an organic solvent. The organic solvent may be a fluorine-based organic solvent, may be a non-fluorine-based organic solvent, or may contain both solvents.

Examples of the fluorine-based organic solvent include fluorinated alkanes, fluorinated aromatic compounds, fluoroalkyl ethers, fluorinated alkylamines, and fluoroalcohols.

The fluorinated alkane is preferably a compound having 4 to 8 carbon atoms. Examples of the commercially available product include C₆F₁₃H (ASAHIKLIN (registered trademark) AC-2000 manufactured by AGC Inc.), C₆F₁₃C₂H₅ (ASAHIKLIN (registered trademark) AC-6000 manufactured by AGC Inc.), and C₂F₅CHFCHFCF₃ (Vertrel (registered trademark) XF manufactured by the Chemours Company).

Examples of the fluorinated aromatic compound include hexafluorobenzene, trifluoromethylbenzene, perfluorotoluene, and bis(trifluoromethyl)benzene.

The fluoroalkyl ether is preferably a compound having 4 to 12 carbon atoms. Examples of the commercially available product include CF₃CH₂OCF₂CF₂H (ASAHIKLIN (registered trademark) AE-3000 manufactured by AGC Inc.), C₄F₉OCH₃ (Novec (registered trademark) 7100 manufactured by 3M), C₄F₉OC₂H₅ (Novec (registered trademark) 7200 manufactured by 3M), and C₂F₅CF(OCH₃)C₃F₇ (Novec (registered trademark) 7300 manufactured by 3M).

Examples of the fluorinated alkylamine include perfluorotripropylamine and perfluorotributylamine.

Examples of the fluoroalcohol include 2,2,3,3-tetrafluoropropanol, 2,2,2-trifluoroethanol, and hexafluoroisopropanol.

As the non-fluorine-based organic solvent, a compound composed only of a hydrogen atom and a carbon atom and a compound composed only of a hydrogen atom, a carbon atom, and an oxygen atom are preferable, and examples thereof include a hydrocarbon-based organic solvent, an alcohol-based organic solvent, a ketone-based organic solvent, an ether-based organic solvent, and an ester-based organic solvent.

The present coating liquid preferably contains 75 to 99.999% by mass, preferably 85 to 99.99% by mass, and particularly preferably 90 to 99.9% by mass of the liquid medium.

The present coating liquid may contain other components in addition to the present compound or the present composition and the liquid medium as long as the effects of the present invention are not impaired.

Examples of other components include known additives such as an acid catalyst and a basic catalyst that promote hydrolysis and condensation reaction of a hydrolyzable silyl group.

The content of other components in the present coating liquid is preferably 10% by mass or less, and more preferably 1% by mass or less.

The total concentration of the present compound and other components or the total concentration of the present composition and other components in the present coating liquid (hereinafter also referred to as a solid content concentration) is preferably 0.001 to 40% by mass, more preferably 0.01 to 20% by mass, still more preferably 0.01 to 10% by mass, and particularly preferably to 1% by mass. The solid content concentration of the coating liquid is a value calculated from the mass of the coating liquid before heating and the mass after heating in a convection dryer at 120° C. for 4 hours.

### [Article]

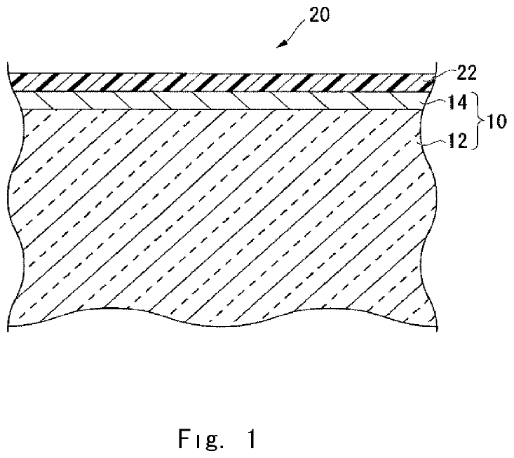

FIG. 1 is a schematic cross-sectional view showing an example of an article of the present invention. A first article of the present invention is an article 20 having a substrate 12, a base layer 14, and a surface layer 22 in this order, in which
the base layer 14 contains an oxide containing silicon, and the surface layer 22 contains a condensate of the present compound.

The material and shape of the substrate 12 in the first article may be appropriately selected according to the application of the present article 20 and the like. Examples of the material of the substrate 12 include glass, resin, sapphire, metal, ceramic, stone, and composite materials thereof. The glass may be chemically strengthened. In particular, examples of the substrate 12 required to have water/oil repellency include a substrate for a touch panel, a substrate for a display, and a substrate constituting a housing of electronic equipment. The substrate for a touch panel and the substrate for a display have translucency. “Having translucency” means that the normal incidence type visible light transmittance according to JIS R3106: 1998 (ISO 9050: 1990) is 25% or more. The material of the substrate for a touch panel is preferably glass or a transparent resin.

The substrate 12 may be obtained by subjecting the surface on which the base layer 14 is provided to a surface treatment such as a corona discharge treatment, a plasma treatment, or a plasma graft polymerization treatment. In the surface-treated surface, the adhesiveness between the substrate 12 and the base layer 14 is further excellent, and as a result, the wear resistance of the surface layer 22 is further improved. As the surface treatment, corona discharge treatment or plasma treatment is preferable from the viewpoint of further excellent wear resistance of the surface layer 22.

The base layer 14 is a layer containing an oxide containing at least silicon, and may further contain other elements. When the base layer 14 contains silicon oxide, T of the present compound is dehydration-condensed, a Si—O—Si bond is formed between the base layer 14 and the base layer, and the surface layer 22 having excellent wear durability is formed.

The content of silicon oxide in the base layer 14 may be 65% by mass or more, and is preferably 80% by mass or more, more preferably 85% by mass or more, and still more preferably 90% by mass or more. When the content of silicon oxide is equal to or more than the lower limit value of the above range, a Si—O—Si bond is sufficiently formed in the base layer 14, and the mechanical characteristics of the base layer 14 are sufficiently secured. The content of silicon oxide is the remainder obtained by subtracting the sum of the total contents of other elements (in the case of oxides, the amount in terms of oxides) from the mass of the base layer 14.

From the viewpoint of durability of the surface layer 22, the oxide in the base layer 14 preferably further contains one or more elements selected from an alkali metal element, an alkaline earth metal element, a platinum group element, boron, aluminum, phosphorus, titanium, zirconium, iron, nickel, chromium, molybdenum, and tungsten. By containing these elements, the bond between the base layer 14 and the present compound is strengthened, and the wear resistance is improved.

When the base layer 14 contains one or more selected from iron, nickel, and chromium, the total content thereof is preferably 10 to 1,100 ppm by mass, more preferably 50 to 1,100 ppm by mass, still more preferably 50 to 500 ppm by mass, and particularly preferably 50 to 250 ppm by mass in terms of a ratio with respect to silicon oxide.

When the base layer 14 contains one or more selected from aluminum and zirconium, the total content thereof is preferably 10 to 2,500 ppm by mass, more preferably 15 to 2,000 ppm by mass, and still more preferably 20 to 1,000 ppm by mass.

When the base layer 14 contains an alkali metal element, the total content thereof is preferably 0.05 to 15% by mass, more preferably 0.1 to 13% by mass, and still more preferably 1.0 to 10% by mass. In addition, examples of the alkali metal element include lithium, sodium, potassium, rubidium, and cesium.

When the base layer 14 contains a platinum group element, the total content thereof is preferably 0.02 ppm by mass or more and 800 ppm by mass or less, more preferably 0.04 ppm by mass or more and 600 ppm by mass or less, and still more preferably 0.7 ppm by mass or more and 200 ppm by mass or less. In addition, examples of the platinum group element include platinum, rhodium, ruthenium, palladium, osmium, and iridium.

When the base layer 14 contains one or more selected from boron and phosphorus, the total content thereof is preferably 0.003 to 9, more preferably 0.003 to 2, and still more preferably 0.003 to 0.5 as the ratio of the total molar concentration of boron and phosphorus to the molar concentration of silicon from the viewpoint of the wear resistance of the surface layer 22.

When the base layer 14 contains an alkaline earth metal element, the total content thereof is preferably 0.005 to 5, more preferably 0.005 to 2, and still more preferably 0.007 to 2 as the ratio of the total molar concentration of the alkaline earth metal element to the molar concentration of silicon from the viewpoint of the wear resistance of the surface layer 22. In addition, examples of the alkaline earth metal element include lithium, sodium, potassium, rubidium, and cesium.

From the viewpoint of improving the adhesiveness of the present compound and improving the water/oil repellency and wear resistance of the article 20, the base layer 14 is preferably a silicon oxide layer containing an alkali metal atom. In particular, in the silicon oxide layer, the average value of the concentrations of alkali metal atoms in the region where the depth from the surface in contact with the surface layer 22 is 0.1 to 0.3 nm is preferably 2.0×10¹⁹ atoms/cm³ or more. On the other hand, from the viewpoint of sufficiently securing the mechanical characteristics of the silicon oxide layer, the average value of the concentrations of the alkali metal atoms is preferably 4.0×10²² atoms/cm³ or less.

The thickness of the base layer 14 is preferably 1 to 200 nm, and particularly preferably 2 to 20 nm. When the thickness of the base layer 14 is equal to or more than the lower limit value of the above range, the effect of improving the adhesiveness by the base layer 14 is easily sufficiently obtained. When the thickness of the base layer 14 is equal to or less than the upper limit value of the above range, the wear resistance of the base layer 14 itself is enhanced. Examples of a method for measuring the thickness of the base layer 14 include a method by observing a cross-section of the base layer 14 with an electron microscope (SEM, TEM, and the like), and a method using an optical interference film thickness meter, a spectroscopic ellipsometer, a step profiler, or the like.

Examples of the method for forming the base layer 14 include a method in which a vapor deposition material having a desired composition of the base layer 14 is vapor-deposited on the surface of the substrate 12.

Examples of the vapor deposition method include a vacuum vapor deposition method. The vacuum vapor deposition method is a method in which a vapor deposition material is evaporated in a vacuum tank and attached to the surface of the substrate 12.

The temperature during vapor deposition (for example, when a vacuum vapor deposition apparatus is used, the temperature of the boat on which the vapor deposition material is installed) is preferably 100 to 3,000° C., and particularly preferably 500 to 3,000° C.

The pressure during vapor deposition (for example, when a vacuum vapor deposition apparatus is used, the absolute pressure in the tank in which the vapor deposition material is installed) is preferably 1 Pa or less, and particularly preferably 0.1 Pa or less.

When the base layer 14 is formed using a vapor deposition material, one vapor deposition material may be used, or two or more vapor deposition materials containing different elements may be used.

Examples of the method for evaporating the vapor deposition material include a resistance heating method of melting and evaporating the vapor deposition material on a resistance heating boat made of a high melting point metal, and an electron gun method of irradiating the vapor deposition material with an electron beam and directly heating the vapor deposition material to melt the surface and evaporate the vapor deposition material. As the method for evaporating the vapor deposition material, the electron gun method is preferable from the viewpoint that a high melting point substance can also be evaporated because the vapor deposition material can be locally heated, and that there is no concern about reaction with a container or mixing of impurities because a part not hit by an electron beam is at a low temperature. The vapor deposition material used in the electron gun method is preferably a molten granular material or a sintered body from the viewpoint of being less likely to scatter even when an air flow is generated.

The surface layer 22 on the base layer 14 contains a condensate of the present compound. The condensate of the present compound includes a compound in which a silanol group (Si—OH) is formed by a hydrolysis reaction of the hydrolyzable silyl group in the present compound, and a Si—O—Si bond is formed by an intermolecular condensation reaction of the silanol group, and a compound in which a Si—O—Si bond is formed by a condensation reaction of the silanol group in the present compound with a silanol group or a Si—OM group (where M is an alkali metal element) on the surface of the base layer 14. In addition, the surface layer 22 may contain a condensate of a fluorine-containing compound other than the present compound. That is, the surface layer 22 contains the fluorine-containing compound having a reactive silyl group in a state where a part or all of the reactive silyl group of the fluorine-containing compound undergoes a condensation reaction.

The thickness of the surface layer 22 is preferably 1 to 100 nm, and particularly preferably 1 to 50 nm. When the thickness of the surface layer 22 is equal to or more than the lower limit value of the above range, the effect of the surface layer 22 can be sufficiently obtained. When the thickness of the surface layer 22 is equal to or less than the upper limit value of the above range, the utilization efficiency is high.

The thickness of the surface layer 22 is a thickness obtained by an X-ray diffractometer for thin film analysis. The thickness of the surface layer 22 can be calculated from the vibration period of the interference pattern by obtaining the interference pattern of the reflected X-ray by the X-ray reflectance method using the X-ray diffractometer for thin film analysis.

A second article of the present invention is the article 20 having a base layer-attached substrate 10 and the surface layer 22 in this order, in which
the base layer-attached substrate 10 contains an oxide containing silicon, and
the surface layer 22 contains a condensate of the present compound.

In the second article, since the base layer-attached substrate 10 has the composition of the base layer 14 in the first article, the wear durability of the surface layer 22 is excellent even when the surface layer 22 is directly formed on the base layer-attached substrate 10.

The material of the base layer-attached substrate 10 in the second article may be any material having the composition of the base layer 14, and may be, for example, a glass substrate or the like. The details of the material of the base layer-attached substrate 10 are the same as the materials of the substrate 12 and the base layer 14, and thus the description thereof will be omitted here. In addition, since the configuration of the surface layer 22 is also similar to that of the first article, the description thereof will be omitted here.

### [Method for Producing Article]

The method for producing an article according to the present invention is a method for forming a surface layer by a dry coating method or a wet coating method using the fluorine-containing compound, the fluorine-containing compound-containing composition, or the coating liquid.

The present compound and the present composition can be used as they are in a dry coating method. The present compound and the present composition are preferred for forming a surface layer excellent in adhesion by a dry coating method. Examples of the dry coating method include techniques such as vacuum vapor deposition, CVD, and sputtering. The vacuum vapor deposition method can be suitably used from the viewpoint of suppressing decomposition of the present compound and from the viewpoint of convenience of the apparatus.

For vacuum vapor deposition, a pellet-like substance that supports the present compound on a metal porous body made of a metal material such as iron or steel may be used. The pellet-like substance that supports the present compound can be produced by impregnating the metal porous body with a solution of the present compound and drying the solution to remove the liquid medium. As the solution of the present compound, the present coating liquid can be used.

The present coating liquid can be suitably used in a wet coating method. Examples of the wet coating method include a spin coating method, a wipe coating method, a spray coating method, a squeegee coating method, a dip coating method, a die coating method, an inkjet method, a flow coating method, a roll coating method, a casting method, a Langmuir-Blodgett method, and a gravure coating method.

In order to improve the friction resistance of the surface layer, an operation for promoting the reaction between the present compound and the substrate may be performed as necessary. Examples of the operation include heating, humidification, and light irradiation. For example, the substrate on which the surface layer is formed can be heated in the atmosphere having moisture to promote reactions such as a hydrolysis reaction of a hydrolyzable group, a reaction between a hydroxyl group or the like on the surface of the substrate and a silanol group, and generation of a siloxane bond by a condensation reaction of a silanol group.

After the surface treatment, the compound in the surface layer, which is a compound that is not chemically bonded to another compound or the substrate, may be removed as necessary. Specific examples of the method include a method in which a solvent is poured on the surface layer and a method in which the surface layer is wiped with a cloth soaked with the solvent.

### EXAMPLES

Hereinafter, the present invention will be described in detail using examples, but the present invention is not limited to these examples. Hereinafter, “%” is “% by mass” unless otherwise specified. In addition, Examples 1, 3, and 16 are production examples (examples) of the compound (B), Examples 2, 4, and 17 are production examples (examples) of the compound (A), Examples 5 to 14 and 18 to 20 are examples, and Example 15 is a comparative example.

### Production Example 1

The following compound 2 was added to 10 g of the following compound 1, and the mixture was stirred at room temperature for 4 hours. The obtained mixture was purified by column chromatography (silica gel 50 g) to obtain 4.6 g of the following compound 3.

H₃CO—C(O)—CF₂CF₂CF₂—(OCF₂CF₂—OCF₂CF₂CF₂CF₂)ₙ—OCF₂CF₂—OCF₂CF₂—CF₂—C(O)OCH₃ Compound 1:

H₂NCH₂C(CH₂CH═CH₂)₃ Compound 2:

H₃CO—C(O)—CF₂CF₂CF₂—(OCF₂CF₂—OCF₂CF₂CF₂CF₂)ₙ—OCF₂CF₂—OCF₂CF₂—CF₂—C(O)—NH—CH₂—C(CH₂—CH═CH₂)₃ Compound 3:

In addition, the average value of n of the compound 1 and the compound 3 is 11.

### Production Example 2

Sodium borohydride was added to 10 g of C₆F₁₃H, and the mixture was stirred. To the obtained mixed solution, a mixture of methanol, 10 g of C₆F₁₃H, and 3 g of the compound 3 was slowly added dropwise. After stirring the mixture at room temperature for 4 hours, methanol was slowly added. Subsequently, 1 M aqueous hydrochloric acid was slowly added and extracted with 20 g of C₆F₁₃H. The organic phase was dried over magnesium sulfate, filtered, and then concentrated. The obtained mixture was purified by column chromatography (silica gel) to obtain 2.5 g of the following compound 4.

HO—CH₂—CF₂CF₂CF₂—(OCF₂CF₂—OCF₂CF₂CF₂CF₂)ₙ—OCF₂CF₂—OCF₂CF₂—CF₂—C(O)—NH—CH₂—C(CH₂—CH═CH₂)₃ Compound 4:

### Production Example 3

The compound 4 (2 g), triphenylphosphine (1 g), 1,3-bistrifluoromethylbenzene (10 mL), carbon tetrabromide (1 g), and dimethylformamide (2 mL) were added, and then the mixture was stirred at 100° C. for 12 hours. After filtration, the solvent was distilled off, and purification was performed by column chromatography (silica gel) to obtain 1.6 g of the following compound 5.

Br—CH₂—CF₂CF₂CF₂—(OCF₂CF₂—OCF₂CF₂CF₂CF₂)ₙ—OCF₂CF₂—OCF₂CF₂—CF₂—C(O)—NH—CH₂—C(CH₂—CH═CH₂)₃ Compound 5:

### Example 1

The compound 5 (1.5 g), 1,3-bistrifluoromethylbenzene (4 mL), acetic acid (2 mL), and zinc (1 g) were added, and then the mixture was stirred at 110° C. for 12 hours. After filtration, the mixture was concentrated and purified by column chromatography (silica gel) to obtain 1.2 g of the following compound 6.

CH₂═CF—CF₂CF₂—(OCF₂CF₂—OCF₂CF₂CF₂CF₂)ₙ—OCF₂CF₂—OCF₂CF₂—CF₂—C(O)—NH—CH₂—C(CH₂—CH═CH₂)₃ Compound 6:

### Example 2

5 (1 g) of the compound 6, C₆F₁₃H, platinum/1,3-divinyl-1,1,3,3, a xylene solution of a -tetramethyldisiloxane complex (platinum content 2%, 5.5 mg), aniline (0.8 mg), and trimethoxysilane (22.7 mg) were added, the mixture was stirred at 40° C. for 5 hours, and then the solvent was distilled off under reduced pressure to obtain 1 g of the following compound 7.

CH₂═CF—CF₂CF₂—(OCF₂CF₂—OCF₂CF₂CF₂CF₂)ₙ—OCF₂CF₂—OCF₂CF₂—CF₂—C(O)—NH—CH₂—C(CH₂CH₂CH₂—Si(OCH₃)₃)₃ Compound 7:

### Example 3

The compound 4 (500 mg), 1,3-bistrifluoromethylbenzene (1 mL), TBAI (5 mg), 30% sodium hydroxide aqueous solution (25 mg), and 3-Bromo-2-fluoroprop-1-ene (70 mg) were added, and then the mixture was stirred at 60° C. for 12 hours. The obtained solution was subjected to an extraction operation using C₆F₁₃H (10 mL), and then the organic phase was dehydrated with magnesium sulfate, filtered, and concentrated. The obtained mixture was purified by column chromatography (silica gel) to obtain 420 mg of the following compound 8.

CH₂═CF—CH₂—O—CH₂—CF₂CF₂CF₂—(OCF₂CF₂—OCF₂CF₂CF₂CF₂)ₙ—OCF₂CF₂—OCF₂CF₂—CF₂—C(O)—NH—CH₂—C(CH₂—CH═CH₂)₃ Compound 8:

### Example 4

(400 mg) of the compound 8, C₆F₁₃H, platinum/1,3-divinyl-1,1,3,3, a xylene solution of a -tetramethyldisiloxane complex (platinum content 2%, 2.2 mg), aniline (0.3 mg), and trimethoxysilane (10.0 mg) were added, the mixture was stirred at 40° C. for 5 hours, and then the solvent was distilled off under reduced pressure to obtain 450 mg of the compound 9.

CH₂═CF—CH₂—O—CH₂—CF₂CF₂CF₂—(OCF₂CF₂—OCF₂CF₂CF₂CF₂)ₙ—OCF₂CF₂—OCF₂CF₂—CF₂—C(O)—NH—CH₂—C(CH₂CH₂CH₂—Si(OCH₃)₃)₃ Compound 9:

In addition, the following compound 10 and compound 11 were prepared. The compound 10 was synthesized by the method described in International Patent Publication No. WO 2017/038830. In addition, the compound 11 was synthesized by the method described in International Patent Publication No. WO 2015/166760.

CF₃—(OCF₂CF₂—OCF₂CF₂CF₂CF₂)ₙ—OCF₂CF₂—OCF₂CF₂—CF₂—C(O)—NH—CH₂—C(CH₂CH₂CH₂—Si(OCH₃)₃)₃ Compound 10:

In addition, the average value of n of the compound 10 is 12.

### Compound 11:

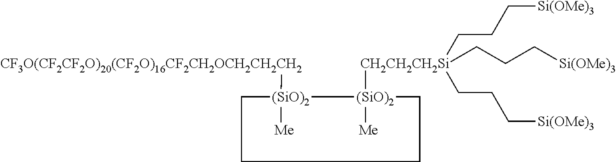

### Examples 5 to 14: Preparation of Fluorine-Containing Ether Composition

The compound 7, the compound 9, and the compound 10 were mixed at a ratio shown in the following Table 1 to obtain a fluorine-containing ether composition.

TABLE 1

| | Example | Example | Example | Example | Example | Example | Example | Example | Example | Example |
|---|---|---|---|---|---|---|---|---|---|---|
| | 5 | 6 | 7 | 8 | 9 | 10 | 11 | 12 | 13 | 14 |
| First | Com- | Com- | Com- | Com- | Com- | Com- | Com- | Com- | Com- | Com- |
| component | pound | pound | pound | pound | pound | pound | pound | pound | pound | pound |
| | 10 | 10 | 10 | 10 | 10 | 10 | 10 | 10 | 10 | 10 |
| Second | Com- | Com- | Com- | Com- | Com- | Com- | Com- | Com- | Com- | Com- |
| component | pound | pound | pound | pound | pound | pound | pound | pound | pound | pound |
| | 7 | 7 | 7 | 7 | 7 | 9 | 9 | 9 | 9 | 9 |
| Content | 99 | 95 | 90 | 80 | 70 | 99 | 95 | 90 | 80 | 70 |
| of first | | | | | | | | | | |
| component | | | | | | | | | | |
| (part by | | | | | | | | | | |
| mass) | | | | | | | | | | |
| Content | 1 | 5 | 10 | 20 | 30 | 1 | 5 | 10 | 20 | 30 |
| of second | | | | | | | | | | |
| component | | | | | | | | | | |
| (part by | | | | | | | | | | |
| mass) | | | | | | | | | | |

### [Production and Evaluation of Article]

A substrate was surface-treated using the fluorine-containing ether composition obtained in Examples 5 to 14 and the compound 11 as Example 15 to produce an article. The following dry coating method was used for the surface treatment. As the substrate, chemically strengthened glass was used. The obtained article was evaluated by the following method. The results are shown in Table 2.

### (Dry Coating Method)

The dry coating was performed using a vacuum vapor deposition apparatus (VTR350M manufactured by ULVAC, Inc.) (vacuum vapor deposition method). A molybdenum boat in a vacuum vapor deposition apparatus was filled with 0.5 g of each of the composition or the compound of Examples 5 to 15, and the inside of the vacuum vapor deposition apparatus was evacuated to 1×10⁻³ Pa or less. The boat on which the composition or compound was disposed was heated at a temperature rise rate of 10° C./min or less, and when the vapor deposition rate by a crystal oscillation type film thickness meter exceeded 1 nm/sec, the shutter was opened to start film formation on the surface of the substrate. When the film thickness reached approximately 50 nm, the shutter was closed to complete film formation on the surface of the substrate. The substrate on which the compound was deposited was heat-treated at 200° C. for 30 minutes and washed with dichloropentafluoropropane (AK-225 manufactured by AGC Inc.) to obtain an article having a surface layer on the surface of the substrate.

### (Evaluation Method)

### <UV Irradiation Condition>

The surface layer was irradiated with a light beam (650 W/m2, 300 to 700 nm) using a desktop xenon arc lamp accelerated light fastness tester (SUNTEST XLS+ manufactured by Toyo Seiki Seisaku-sho, Ltd.) at a black panel temperature of 63° C.

### <Method for Measuring Contact Angle>

The contact angle of approximately 2 μL of distilled water or n-hexadecane placed on the surface of the surface layer was measured using a contact angle measurement apparatus (DM-500 manufactured by Kyowa Interface Science Co., Ltd.). Measurement was performed at five different positions on the surface of the surface layer, and the average value thereof was calculated.

The 2θ method was used to calculate the contact angle. The measurement was performed every 30 minutes of UV irradiation time. The results are shown in Table 2. It can be evaluated that the larger the numerical value, the better the water repellency, and the smaller the decrease amount of the numerical value, the better the light fastness.

TABLE 2

| Exposure | Contact angle (degree) | | | | | | | | | | |
|---|---|---|---|---|---|---|---|---|---|---|---|
| time | Example | Example | Example | Example | Example | Example | Example | Example | Example | Example | Example |
| (time) | 5 | 6 | 7 | 8 | 9 | 10 | 11 | 12 | 13 | 14 | 15 |
| 0 | 115 | 115 | 115 | 114 | 114 | 115 | 115 | 114 | 114 | 112 | 114 |
| 30 | 115 | 115 | 115 | 114 | 114 | 115 | 115 | 114 | 114 | 111 | 113 |
| 60 | 115 | 115 | 114 | 114 | 113 | 114 | 114 | 113 | 113 | 111 | 112 |
| 90 | 114 | 115 | 114 | 113 | 113 | 113 | 114 | 113 | 112 | 110 | 111 |
| 120 | 113 | 114 | 113 | 112 | 112 | 112 | 113 | 112 | 111 | 109 | 110 |
| 150 | 112 | 114 | 112 | 110 | 110 | 111 | 112 | 110 | 110 | 108 | 109 |
| 180 | 111 | 113 | 110 | 110 | 110 | 109 | 111 | 110 | 110 | 107 | 107 |
| 210 | 110 | 112 | 110 | 108 | 108 | 109 | 109 | 108 | 109 | 106 | 105 |
| 240 | 109 | 110 | 108 | 107 | 106 | 108 | 108 | 107 | 107 | 104 | 104 |
| 270 | 108 | 109 | 107 | 106 | 105 | 107 | 106 | 105 | 105 | 102 | 103 |
| 300 | 106 | 108 | 105 | 104 | 103 | 105 | 104 | 104 | 103 | 100 | 101 |

As shown in Table 2, the surface layer formed using the fluorine-containing ether composition containing the compound (A) was found to be excellent in light fastness.

### Production Example 4

The following compound 2 was added to 10 g of the following compound 20, and the mixture was stirred at room temperature for 4 hours. The obtained mixture was purified by column chromatography (silica gel 50 g) to obtain 4.6 g of the following compound 21.

H₃CO—C(O)—CF₂—(OCF₂)ₘ—(OCF₂CF₂)ₙ—OCF₂—C(O)OCH₃ Compound 20:

H₂NCH₂C(CH₂CH═CH₂)₃ Compound 2:

H₃CO—C(O)—CF₂—(OCF₂)ₘ—(OCF₂CF₂)ₙ—OCF₂—C(O)—NH—CH₂—C(CH₂—CH═CH₂)₃ Compound 21:

In addition, the average value of m of the compound 20 and the compound 21 is 21, and the average value of n is 19.

### Production Example 5

Sodium borohydride was added to 10 g of C₆F₁₃H, and the mixture was stirred. To the obtained mixed solution, a mixture of methanol, 10 g of C₆F₁₃H, and 3 g of the compound 21 was slowly added dropwise. After stirring the mixture at room temperature for 4 hours, methanol was slowly added. Subsequently, 1 M aqueous hydrochloric acid was slowly added and extracted with 20 g of C₆F₁₃H. The organic phase was dried over magnesium sulfate, filtered, and then concentrated. The obtained mixture was purified by column chromatography (silica gel) to obtain 2.5 g of the following compound 22.

HO—CH₂—CF₂—(OCF₂)ₘ—(OCF₂CF₂)ₙ—OCF₂—C(O)—NH—CH₂—C(CH₂—CH═CH₂)₃ Compound 22:

### Production Example 6

The compound 22 (2 g), triphenylphosphine (1 g), 1,3-bistrifluoromethylbenzene (10 mL), carbon tetrabromide (1 g), and dimethylformamide (2 mL) were added, and then the mixture was stirred at 100° C. for 12 hours. After filtration, the solvent was distilled off, and purification was performed by column chromatography (silica gel) to obtain 1.6 g of the following compound 23.

Br—CH₂—CF₂—(OCF₂)ₘ—(OCF₂CF₂)ₙ—OCF₂—C(O)—NH—CH₂—C(CH₂—CH═CH₂)₃ Compound 23:

### Example 16

The compound 23 (1.5 g), 1,3-bistrifluoromethylbenzene (4 mL), acetic acid (2 mL), and zinc (1 g) were added, and then the mixture was stirred at 110° C. for 12 hours. After filtration, the mixture was concentrated and purified by column chromatography (silica gel) to obtain 1.2 g of the following compound 24.

CH₂═CF—(OCF₂)ₘ—(OCF₂CF₂)ₙ—OCF₂—C(O)—NH—CH₂—C(CH₂—CH═CH₂)₃ Compound 24:

### Example 17

(1 g) of the compound 24, C₆F₁₃H, platinum/1,3-divinyl-1,1,3,3, a xylene solution of a -tetramethyldisiloxane complex (platinum content 2%, 5.5 mg), aniline (0.8 mg), and trimethoxysilane (22.7 mg) were added, the mixture was stirred at 40° C. for 5 hours, and then the solvent was distilled off under reduced pressure to obtain 1 g of the following compound 25.

CH₂═CF—(OCF₂)ₘ—(OCF₂CF₂)ₙ—OCF₂—C(O)—NH—CH₂—C(CH₂CH₂CH₂—Si(OCH₃)₃)₃ Compound 25:

### Examples 18 to 20: Preparation of Fluorine-Containing Ether Composition

The compound 25 and the compound 10 were mixed at a ratio shown in the following Table 3 to obtain a fluorine-containing ether composition.

TABLE 3

| | Example 18 | Example 19 | Example 20 |
|---|---|---|---|
| First component | Compound 10 | Compound 10 | Compound 10 |
| Second component | Compound 25 | Compound 25 | Compound 25 |
| Content of first | 99 | 95 | 90 |
| component (part by | | | |
| mass) | | | |
| Content of second | 1 | 5 | 10 |
| component (part by | | | |
| mass) | | | |

The substrate was surface-treated in the same manner as in Example 5 described above to produce an article. The dry coating method was used for the surface treatment. As the substrate, chemically strengthened glass was used. The obtained article was evaluated by the UV irradiation condition and the method for measuring the contact angle. The results are shown in Table 4.

TABLE 4

| Exposure | Contact angle (degree) | | |
|---|---|---|---|
| time (time) | Example 18 | Example 19 | Example 20 |
| 0 | 115 | 115 | 115 |
| 30 | 115 | 115 | 115 |
| 60 | 115 | 115 | 114 |
| 90 | 114 | 115 | 114 |
| 120 | 114 | 114 | 114 |
| 150 | 113 | 114 | 113 |
| 180 | 112 | 113 | 113 |
| 210 | 111 | 112 | 112 |
| 240 | 110 | 110 | 112 |
| 270 | 109 | 110 | 110 |
| 300 | 108 | 109 | 108 |

As shown in Table 4, the surface layer formed using the fluorine-containing ether composition containing the compound (A) was found to be excellent in light fastness.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A fluorine-containing ether compound represented by the following general formula (A):
(R¹R²C═CR³-L¹-)ₙ₁Q¹-R^{f}-Q²(-T)ₙ₂ Formula (A)
where
R¹ and R² are each independently a hydrogen atom or an alkyl group which may have a substituent, and when there are a plurality of R¹'s and R²'s, the plurality of R¹'s and R²'s may be the same as or different from each other,
R³ is a hydrogen atom, a halogen atom, or an alkyl group which may have a substituent, and when there are a plurality of R³'s, the plurality of R³'s may be the same as or different from each other,
L¹ is an oxygen atom or CR⁴R⁵, and when there are a plurality of L¹'s, the plurality of L¹'s may be the same as or different from each other,
R⁴ and R⁵ are each independently a hydrogen atom, a halogen atom, or an alkyl group which may have a substituent, and when there are a plurality of R⁴'s and R⁵'s, the plurality of R⁴'s and R⁵'s may be the same as or different from each other,
R^{f} is a polyfluoropolyether chain,
Q¹ is an (n1+1)-valent linking group,
Q² is an (n2+1)-valent linking group,
T is Si(—R⁶)₃₋ₐ(—R⁷)ₐ, and when there are a plurality of T's, the plurality of T's may be the same as or different from each other,
R⁶ is a hydrogen atom or a hydrocarbon group, and when there are a plurality of R⁶'s, the plurality of R⁶'s may be the same as or different from each other,
R⁷ is a hydrolyzable group or a hydroxyl group, and when there are a plurality of R⁷'s, the plurality of R⁷'s may be the same as or different from each other,
a is an integer of 1 to 3,
n1 is an integer of 1 to 20, and
n2 is an integer of 1 to 20.

2. A surface treatment agent containing the fluorine-containing ether compound according to claim 1.

3. A fluorine-containing ether composition containing the fluorine-containing ether compound according to claim 1 and another fluorine-containing ether compound.

4. A coating liquid containing
the fluorine-containing ether compound according to claim 1, and
a liquid medium.

5. A coating liquid containing
the fluorine-containing ether composition according to claim 3, and
a liquid medium.

6. An article having a surface layer formed from the fluorine-containing ether compound according to claim 1.

7. An article having a surface layer formed from the fluorine-containing ether composition according to claim 3.

8. A method for producing an article, comprising:
forming a surface layer by a dry coating method or a wet coating method using the fluorine-containing ether compound according to claim 1.

9. A method for producing an article, comprising:
forming a surface layer by a dry coating method or a wet coating method using the surface treatment agent according to claim 2.

10. A method for producing an article, comprising:
forming a surface layer by a dry coating method or a wet coating method using the fluorine-containing ether composition according to claim 3.

11. A method for producing an article, comprising:
forming a surface layer by a dry coating method or a wet coating method using the coating liquid according to claim 4.

12. A compound represented by the following general formula (B):
(R¹R²C═CR³-L¹-)ₙ₁Q¹-R^{f}-Q¹²(-L¹¹_ R¹³C═CR¹²R¹¹)ₙ₂ Formula (B)
where
R¹ and R² are each independently a hydrogen atom or an alkyl group which may have a substituent, and when there are a plurality of R¹'s and R²'s, the plurality of R¹'s and R²'s may be the same as or different from each other,
R³ is a hydrogen atom, a halogen atom, or an alkyl group which may have a substituent, and when there are a plurality of R³'s, the plurality of R³'s may be the same as or different from each other,
L¹ is an oxygen atom or CR⁴R⁵, and when there are a plurality of L¹'s, the plurality of L¹'s may be the same as or different from each other,
R⁴ and R⁵ are each independently a hydrogen atom, a halogen atom, or an alkyl group which may have a substituent, and when there are a plurality of R⁴'s and R⁵'s, the plurality of R⁴'s and R⁵'s may be the same as or different from each other,
R¹¹ and R¹² are each independently a hydrogen atom or an alkyl group which may have a substituent, and when there are a plurality of R¹¹'s and R¹²'s, the plurality of R¹¹'s and R¹²'s may be the same as or different from each other,
R¹³ is a hydrogen atom, a halogen atom, or an alkyl group which may have a substituent, and when there are a plurality of R¹³'s, the plurality of R¹³'s may be the same as or different from each other,
L¹¹ is an oxygen atom or CR¹⁴R¹⁵, and when there are a plurality of L¹¹'s, the plurality of L¹¹'s may be the same as or different from each other,
R¹⁴ and R¹⁵ are each independently a hydrogen atom, a halogen atom, or an alkyl group which may have a substituent, and when there are a plurality of R¹⁴'s and R¹⁵'s, the plurality of R¹⁴'s and R¹⁵'s may be the same as or different from each other,
R¹R²C═CR³-L¹- and R¹¹R¹²C═CR¹³-L¹¹- are different structures,
R^{f} is a polyfluoropolyether chain,
Q¹ is an (n1+1)-valent linking group,
Q¹² is an (n2+1)-valent linking group,
n1 is an integer of 1 to 20, and
n2 is an integer of 1 to 20.
